(19) United States
(12) Patent Application Publication
(10) Pub. No.: US 2016/0287545 A1
(43) Pub. Date: Oct. 6, 2016

(54) COMPOUND PARTICULARLY FOR TREATING DEPRESSION AND ANXIETY

(71) Applicants: Guerrino ZAGGIA, SELVAZZANO DENTRO (IT); Matteo BEVILACQUA, PADOVA (IT)

(72) Inventor: Matteo BEVILACQUA, PADOVA (IT)

(21) Appl. No.: 14/442,654

(22) PCT Filed: Nov. 13, 2013
(86) PCT No.: PCT/IB2013/060106

§ 371 Date: May 13, 2015

(30) Foreign Application Priority Data
Nov. 13, 2012 (IT) PD2012A000343

(51) Int. Cl. A61K 31/22; A61K 31/015; A61K 31/35; A61K 31/19

(52) U.S. Cl. CPC A61K 31/22; A61K 31/19; A61K 31/015; A61K 31/35

(57) ABSTRACT

A compound particularly for treating depression and anxiety, having the combination of at least 11-keto-beta-boswellic acid (KBA) and acetyl-11-keto-beta-boswellic acid (AKBA). Various doses of the compound are also used in particular physiopathologies.

### FIELD

The present disclosure relates to a compound particularly for treating depression and anxiety.

### BACKGROUND

### Depression and Anxiety

### Definitions and Prevalences

States of depression are among the most frequently underrated diagnoses in clinical practice.

Depression probably causes far more suffering than any other disease that can affect mankind⁽¹⁾.

The various forms of depression, taken all together, are the most frequent psychiatric disorders (50% of psychiatric consultations, 12% of all admissions).

The term “depression” does not indicate only a sense of sadness or unhappiness. It references a complex of altered states of mind (defined as mood or affection disorders) that can include desperation, sense of uselessness, self-destructive thoughts associated with a reduction in energy and libido, loss of interest for life, reduction in concentration, various alterations in thought and behavior and clear physical disorders, the most important of which are insomnia, anorexia or bulimia, cephalea and a variety of localized pains.

At one extreme there are psychotic depressive manifestations (including paranoia and somatic deliriums), which wreak havoc in the life of the patient and in the life of those who are next to him; at the other extreme there are sensations of unhappiness, anhedonia (absence of pleasure in performing acts that usually are pleasurable), dejection and rancor, which become apparent in almost all subjects as a reaction to the disappointments of everyday life, such as for example loss of work, lack of appreciation, an unsatisfactory sex life or social life, all of which are tightly linked to the persistence of triggering factors⁽²⁾.

Together with depression, anxiety is among the symptoms most frequently observed in outpatient and hospital practice. A study conducted in the United Kingdom pointed out that more than 40% of the population has severe symptoms linked to anxiety in a period of life and approximately 5% suffers from states of anxiety that last all of their life. The enormous quantities of anxiolytic drugs and of alcohol consumed in our society tend to support these data⁽³⁾.

Anxiety is defined as an intermittent or prolonged emotional state characterized by a subjective sense of nervousness, irritability, unpleasant anticipation and apprehension, generally with a specific topical contact (i.e., the idea, the person or the object regarding which the person is anxious) associated with accompanying physiological phenomena of intense emotion (dyspnea, sense of chest oppression, sense of suffocation, palpitations, increased muscle tension, sense of chest constriction, confusion, tremors, sweating and hot flushes)⁽³⁾.

The close association of depression with anxiety has been recognized for a long time both by clinicians and by researchers and has triggered at the lively and persistent dispute between so-called “separators” and so-called “unifiers”, i.e., between those who believe that depression and anxiety are two separate category entities and those who support the unitary hypothesis, considering both as dimensions of a single underlying disorder.

The tight bond between anxiety and depression is reflected perfectly in the overlap of the items related to anxiety and depression in the scales most widely used to assess the severity of the two disorders.

In the current state of research, it appears that there can be no doubt as to the fact that the simultaneous onset of depression and anxiety is the rule rather than the exception, and this occurs most of all in general medicine environments⁽⁴⁾.

### Particular Clinical Aspects of Depression

### 1. Anorexia Nervosa and Bulimia.

Weight loss, lack of self-esteem and of interest for one's own appearance and self-destructive behavior—typical characteristics of anorexia nervosa—are certainly also a symptom of depression, but most female patients do not appear to be dejected and do not report being depressed. Some psychiatrists have reported a percentage of successes when the administration of imipramine or fluoxetine is added to psychotherapy and to nasogastric feeding; others have observed that these drugs are effective only in patients with conspicuous symptoms of depression.

The few male adolescents in whom this syndrome has been observed have been cured with an antidepressant treatment.

Bulimia is characterized by massive ingestion of food, followed by induction of vomiting and by excessive use of laxatives. It is considered a variation of anorexia. Pope et al.⁽⁵⁾ have achieved a considerable success in 19 out of 20 patients with bulimia treated with imipramine and followed on for two years.

The antidepressants of the latest generation are equally effective: in general, these drugs are more effective in cases of bulimia than in those of anorexia nervosa⁽²⁾.

### 2. Chronic Fatigue or Chronic Tiredness Syndromes or Neurasthenia or Neurocirculatory Asthenia.

The criteria currently used for diagnosis of this syndrome consist of the presence of persistent and invalidating fatigue for at least 6 months, associated with persistent or recurring somatic and neuropsychological symptoms, including slight fever, cervical or axillary lymphadenopathy, myalgias, migrating arthralgias (association with an equally obscure entity, painful fibromyalgia, is known), sore throat, difficulty in concentration, forgetfulness, cephalea, vertigo, irritability, anxiety, depression, sleep disorders, reduced sexual desire and loss (or sometimes increase) of appetite.

In one study, 85% of the patients visited by a psychiatrist for chronic fatigue were diagnosed with an anxious-depressive syndrome or an anxious neurosis. Likewise, in a more recent study, Wessely and Powell⁽⁶⁾ observed that 72% of patients visited in a neurological center for an inexplicable “state of chronic fatigue” were affected by a psychiatric disorder, more frequently of a depressive type.

The sense of fatigue interferes both with mental activities and with physical activities: the patient worries easily, is mentally scarcely active, is “full of annoyances” and finds it difficult to concentrate to solve a problem, read a book or follow a conversation. Moreover, also sleep is disturbed, with a tendency to wake up early in the morning, a fact which explains why these people feel worse on a morning, both in terms of mood and in terms of energy; they tend to improve as the day goes by and in the evening may even feel well. It is difficult to assess whether fatigue is the primitive manifestation of the disorder or whether it is secondary to loss of interest.

Despite these symptoms, the patient appears rested and in good health and neurological objectivity is negative. These patients often complain of muscle weakness but have normal neuromuscular activity.

The frequency with which a similar syndrome has become, in the United States, the basis for legal actions against employers (“sick building” syndrome) or against the state⁽³⁾ is worth noting.

### 3. Depression and Sexuality.

### a) Depression in Men

Hyposexuality.

Hyposexuality, i.e., loss of libido, is due more often to depression. Nonetheless, some drugs also can cause a reduction in libido, particularly antihypertensives, anti-epileptics, serotoninergic antidepressants and neuroleptics.

Impotence.

Impotence, i.e., the inability to maintain an erection that is sufficient for sexual intercourse, is defined more appropriately as erectile dysfunction.

In the United States, in the general population, approximately half of otherwise healthy men of age between 40 and 70 have erectile dysfunction. It has neither a single “organic” cause nor a single “functional” but is usually caused by a combination of factors, including alcohol abuse, smoking habit, diabetes mellitus, arterial hypertension, the taking of antihypertensive or psychotropic drugs, marital problems, performance anxiety, self-esteem problems and psychiatric disorders, particularly depression.

Although a depressed patient feels sexual desire, there is a general blocking of central and peripheral neurotransmitters, with consequent arousal inability. The release of smooth muscles is the key element in achieving an erection. The administration of prostaglandins also may release penile muscle and produce an erection.

### b) Depression in Women.

There is an increase in the risk of recurrence of a more severe depressive episode whenever, in the course of her life, a woman is in a period of variation of estrogen levels, a phenomenon that some experts have termed “kindling”.

A woman affected by a depressive episode triggered by any endocrine modification is far more vulnerable to recurrence of depression after another subsequent reproductive “event”, such as puberty, pregnancy termination (spontaneous or voluntary), postpartum, perimenopause, taking oral contraceptives and hormone replacement therapy, especially if progestin-based.

Infertility and Depression.

Stress and anxiety can reduce fertility in women. Infertility can be experienced in a woman as a frustrating experience which carries with it a high level of anxiety and depression that can be compared to that induced by severe chronic disorders.

Postpartum Depression.

After childbirth, over 70% of women suffer from a slight form of depression, while 10% suffer from true depression.

Perimenopausal Depression.

Perimenopause is a truly high risk of depression (depressed mood, anhedonia, sense of guilt and uselessness, agitation, slowing, suicidal ideas) due to the irregularity of estrogen cycles, which can last up to 6 years before menopause.

The hormone level can be chaotic and unpredictable and these fluctuations are experienced as physiological and psychological stress factors. Vasomotor phenomena can be the precursors of the onset or relapse of depression and their link is explained by the fact that both are regulated by the trimonoaminergic neurotransmission system. Estrogens in fact modulate the trimonoamine transmission system, regulating gene expression for many receptors of neurotransmitters, synthesizing or metabolizing enzymes.

A deficit in neurotransmitters triggers depression; a loss of neurotransmitter regulation triggers vasomotor symptoms. This justifies the use of antidepressants to treat both depression and vasomotor symptoms in menopause.

Some mental disorders, including recurring depression, may be potentially harmful for the brain due to excitotoxic brain damage. Perhaps modifications in the levels of estrogens over the course of life are a cause of excitotoxicity, indeed as they appear to do at each menstrual cycle⁽⁷⁾.

### 4. Depression and Neurological Disorders

Depression occurs frequently in patients with neurological disorders, especially in those with cerebrovascular disorders affecting the frontolateral dorsal cortex; with degenerative diseases of the nervous system, such as Alzheimer's disease, Huntington's chorea, Parkinson's disease, amyotrophic lateral sclerosis; with demyelinating diseases, such as multiple sclerosis; with muscle dystrophies, cranial traumas, neoplasms, schizophrenia.

Depression is often the main manifestation of a disease that endangers the life and autonomy of the patient. In particular:

### 4.1 Parkinson's Disease.

Depression is a very frequent disorder and is estimated to occur in approximately 40% of patients.

Depressive symptoms or panic attacks can precede the onset of motor symptoms in at least 30% of cases. The most common symptoms are loss of initiative and self-esteem. Cognitive involvement, mnesic difficulties, difficulties in concentration and judgment are often common both to Parkinson's disease and to depression: therefore, the difficulty of a differential diagnosis is evident.

The presence of depression was considered recently a risk factor for a more rapid progression of the disease. Another risk factor is the tendency of L-dopa itself to produce, in a limited number of patients, depression, suicidal tendencies, paranoid ideation and psychotic episodes⁽²⁾.

### 4.2 Multiple Sclerosis.

Depression is mostly a reaction to the disease and to the disability rather than a symptom of disease.

Surrige (1969) observes moderate depression in 17% of patients, moderately severe depression in 7% and severe in 4%. 4% were euphoric and 7% showed conspicuous mood swings. Doubt remains as to whether depression is or not the direct consequence of brain plaques⁽⁸⁾.

### 4.3 Alzheimer's Disease.

Alzheimer's disease also can be accompanied by depressive symptoms, in which case it becomes difficult or even impossible to assess the relative contribution of the mood disorder and dementia in the initial stages of the disease⁽²⁾.

### 5. Depression and Invalidating Chronic Internistic Disorders.

Depressive syndrome influences the etiology, course and final outcome of chronic pathologies, such as cardiovascular, renal, bone and joint pathologies, neuromuscular pathologies and obesity: the association appears to depend on a mutual enhancement with an increased prevalence of these chronic diseases.

It is likely that the onset of depression after a stroke or after myocardial infarction is a reaction to invalidity, i.e., a reactive depression. For example, depression facilitates the onset of cardiovascular pathologies (a depressed person tends to smoke more and to be more sedentary) and reduces the likelihood that after a heart attack or stroke the person will change his or her lifestyle and comply with treatment.

However, the opposite is also true: for several weeks or months after myocardial infarction, some patients report an absolutely disproportionate fatigue with respect to the effort; approximately ⅙ of former infarction patients suffers from major depression and at least twice as many report symptoms of depression. Anxiety is also observed in many patients. Fatigue that can precede myocardial infarction is more difficult to understand.

Almost 50% of people affected by tumors suffer from depression that influences directly survival duration and quality of life; moreover, only a minority receives a targeted pharmacological or psychotherapeutic treatment. All this has important implications for the management and treatment of the depressed subject.

A prevalence of depression that can vary from 8.5% to 27.3% is reported in diabetes mellitus; the severity of mood depression is correlated to the physical symptoms of the pathology and at the level of hyperglycemia.

Clinical hypothyroidism is often associated with depressive symptoms, more frequently depressed mood and memory deficit. Hypothyroid states also can be manifested in similar manners, especially in the elderly⁽⁹⁾.

50-65% of asthmatics have substantial depressive symptoms, partly attributed to the stress caused by the disease, especially in the presence of symptoms such as insomnia and nocturnal dyspnea⁽¹⁰⁾.

Patients with chronic obstructive pulmonary disease (COPD) have an incidence of depression that is estimated at 25%⁽¹¹⁾ to 42% and depression influences the course of the disease and their quality of life⁽¹⁰⁾.

### 6. Depression and Obstructive Sleep Apnea Syndrome (OSAS)

In OSAS sustained mostly by snoring, depression is a very frequent symptom.

The neurobiology of the waking state is linked to an excitatory system that uses the five neurotransmitters histamine, dopamine, noradrenaline, acetylcholine and serotonin as components of the ascending reticular activating system.

Sleep and wake are also regulated by a hypothalamic sleep/wake switch with wake-promoter neurons in the tuberomammillary nucleus that uses histamine as a neurotransmitter and with sleep-promoter neurons in the ventrolateral preoptic nucleus which uses GABA as neurotransmitter.

Sleep-regulating centers of the encephalic trunk, in particular by means of the 5-HT2A postsynaptic receptors, regulate sleep, especially slow-wave sleep; serotoninergic neurons that have their branches in the spinal cord might be involved in control of spinal reflexes that are part of sexual response, such as orgasm and ejaculation; low sexual desire is believed to be due to hypoactivity of mesolimbic dopaminergic neurons⁽⁷⁾.

The more immediate nocturnal physiopathological consequences of OSAS are: sleep fragmentation, intermittent hypoxia and hypercapnia, gastroesophageal reflux. These alterations can cause a severe impairment of the quality of life, with pulmonary and systemic arterial hypertension, cardiac arrhythmias, increased incidence of cardiovascular and cerebrovascular pathology, excessive daytime sleepiness, mood tone disorders (depression, apathy, anxiety, irritability), cognitive deficits, chronic tiredness, reduction in libido and erectile dysfunction.

There are various hypotheses for explaining sexual disorders in OSAS:

1)—apneas induce a drop in the male sex hormone, testosterone, and one of the most evident consequences is erectile dysfunction;
2)—patients with sleep apneas rarely achieve and maintain deep sleep (the so-called REM phase), which is the phase of sleep in which men have erections: erections during sleep are fundamental for the health and oxygenation of the penis and erectile dysfunction is almost a certainty if they are absent;
3)—the rhythmic drop in oxygen caused by apneas leads to an altered metabolism of sex hormones;
4)—sleep interrupted rhythmically by apneas undergoes deterioration and this leads to fatigue, tiredness and reduction in libido. The higher the severity of the apnea syndrome, the higher the severity of the erectile dysfunction.

### 7. Iatrogenic Depression

Alcoholism, sedative drugs, anti-tubercular drugs, beta-blocking agents, beta interferon, phenothiazines, oral contraceptives can evoke a depressive reaction.

Corticosteroids can induce a peculiar psychiatric state in which confusion, insomnia and an elevated mood tone, depression are associated, i.e., a hypomaniac state.

### Particular Clinical Aspects of Anxiety

### 1. Persistent Anxiety and Anxious Depression

Protracted episodic anxiety, without a mood disorder (i.e., without depression), is classified as anxiety disorder or anxiety neurosis.

However, the symptoms of anxiety can be part of many other psychiatric disorders, such as hysteria and phobic neurosis.

Inexplicable anxiety or panic attacks can sometimes anticipate the onset of a schizophrenic disorder.

Persistent anxiety with insomnia, tiredness and fatigue, independently of mood, must always lead to suspect the presence of a depressive disease, especially when it begins at middle age or later.

As in asthenia, anxiety and depression are preeminent in posttraumatic nervous instability (post-commotion syndrome) and in posttraumatic stress disorder⁽³⁾.

### 2. Neuroses

Neuroses are the least known, although they are considered the most frequent mental disorders. They were defined as clinical entities at the end of the nineteenth century, but there are still important and unresolved problems regarding their nature, classification and etiology.

In descriptive terms, neuroses comprise the following clinical syndromes:

1)—anxiety neurosis;
2)—phobic neurosis, which includes disease phobia, social phobia, agoraphobia, etc.;
3)—obsessive-compulsive neurosis;
4)—hysteria;
5)—hypochondria.

In more recent classifications, all neuroses have been again replaced with three broad categories of disorders:

1) anxiety disorders (which comprise states of panic, with or without agoraphobia, and phobic and obsessive-compulsive neuroses);
2) Somatization disorders (which include hysterical neuroses, or conversion disorders, and hypochondria);
3) dissociative disorders.

If there is a fundamental characteristic of neuroses, it is believed that it must be anxiety, which occurs as a recurring theme in all forms. Even in hysterical neurosis, in which the patient appears to be indifferent to his own functional disorder, there is a strong hidden component of anxiety⁽²⁾.

### 3. Anxiety Neurosis and Panic Attacks

The expression “anxiety neurosis” was introduced by Freud at the end of the nineteenth century to describe a syndrome characterized by irritability, anxious expectation, anxiety attacks, somatic equivalents of anxiety and nightmares.

In anxiety neuroses, the entire disorder constituted by this set of symptoms; however, as mentioned above, some elements of this syndrome can be part of various other psychiatric disorders: manic-depressive psychosis, schizophrenia, hysteria and phobic neurosis.

Its closest ties are with depression, which resembles it also in another aspect, i.e., important hereditary factors.

The definition used by DSM-IV (Diagnostic and Statistical Manual of Mental Disorders) is anxiety disorders, of which phobias, obsessive-compulsive disorders, panic attacks and so-called posttraumatic stress disorders are important categories.

The symptoms of depression are often added to those of anxiety neurosis and most patients with depression have anxiety symptoms.

Indeed, many psychiatrists believe that anxiety neurosis is only a variation of depression and a state of anxiety that occurs for the first time after 40 years of age usually is depression.

The observation of symptoms such as reduced self-esteem, excessive fatigue, feelings of desperation and ideas of self-destruction in an anxious patient should always suggest depression.

Schizophrenia also can begin with anxiety disturbances, as well as hysteria and obsessive-compulsive phobic neurosis, but each one of these conditions has other distinctive characteristics.

Panic attacks are a widespread disorder (they affect 1 to 2% of the population at least once in the course of life).

The symptoms of anxiety may become manifest with acute episodes, each of which lasts a few minutes, or with a protracted state which can last weeks, months or years.

In acute attacks (panic attacks), patients are suddenly overwhelmed by a feeling of apprehension, of fear of fainting or dying, of having a heart attack or a stroke, of losing their rationality and self-control, by the fear of becoming mad or of committing some horrible crime.

These experiences are accompanied by a series of physiological reactions, especially linked to hyperactivity of the sympathetic-adrenal type, which resemble the “fight or flight” reaction.

Dyspnea, feeling of suffocation, dizziness, sweating, tremors, paresthesia, palpitations, gastric discomfort or precordialgia are the most typical accompanying somatic symptoms, although they do not always occur.

In less severe and more persistent cases, the patient reports fluctuating degrees of nervousness, palpitations or a feeling of increased intensity of heartbeats, shortness of breath, lightheadedness or feeling of imminent fainting, weakness, easy fatigue and intolerance of physical effort.

Generally, apprehension and somatic symptoms increase in intensity over a few minutes and then decrease within 20-30 minutes.

Panic crises and a state of permanent anxiety often overlap.

The fear of further panic attacks induces in many patients, especially women, the onset of agoraphobia (i.e., fear of public spaces), especially when alone.

The symptoms tend to occur periodically and begin between the ages of 20 and 30; a later onset is associated with a higher likelihood of depression.

In some cases, this pathological condition is twice as frequent in women as in men and familial incidence is high.

In research conducted by Wheeler et al.⁽¹²⁾, there was a 49% prevalence in children of patients with anxiety neurosis against a 5% prevalence in general population.

Chronic anxiety neurosis arises predominantly in the elderly, especially as part of an “anxious depression” that can be established on the basis of a slight anxiety and obsessiveness that have been present throughout their lives.

Treatment of depression with one of the many available antidepressants (usually one proceeds by trial and error) and careful use of anxiolytics are relatively effective in controlling panic attack disorder⁽³⁾.

### 4. Obsessive-Compulsive Neurosis

These patients often have anxiety attacks; when this condition persists for a long time, they can fall into depression. Easy fatigue, anorexia and general lack of interests, which are often present, are probably phenomena that are secondary to anxiety and depression.

Lesions in the frontal and temporal cingulate cortex, in basal ganglia and the posterior putamen have been observed.

Drugs that inhibit serotonin reuptake, such as fluoxetine, have shown a certain therapeutic effectiveness.

### 5. Phobic Neurosis

In this condition, patients are overwhelmed by intense and irrational fear of an animal, object, social situation or disease, of finding themselves closed in confined spaces (claustrophobia) or in open spaces among people, feeling defenseless (agoraphobia) or at great heights (in the mountains or in an aircraft).

In many patients a phobic neurosis (or obsessive-compulsive neurosis) has become unbalanced under the influence of endogenous depression; recovery from the depressive episode returned them to the condition of slight phobic alarm. This suggests a link between these neuroses and depression.

### 6. Stress and Stress-Related Syndromes

The psychological phenomenon of stress is tied closely to nervousness, fatigue and anxiety, all of which are emotional events that pervade modern life.

Stress has been defined as a sense of personal insecurity regarding one's own ability to bear a situation for a certain period of time.

The expression “stress-related syndrome” refers to behavioral disorders and vegetative alterations that can be ascribed to challenges posed by the surrounding environment, of such intensity and duration as to overwhelm the adaptive abilities of the individual.

Human beings forced to work in confined spaces or in conditions of constant danger lose their ability to bear and become anxious and depressed.

Presumably, a higher release of “stress hormones” (cortisol and adrenaline) occurs in them⁽³⁾.

### Biochemical Etiopathogenic Theories

### a) Depression

It has been known for several years that biogenic monoamines (noradrenaline, serotonin, dopamine) are in some way involved in the biology of depression.

However, most neurochemical theories of depression have the limitation that they are the result of reverse reasoning, which starts from the biochemical effects of antidepressants on several neurotransmitters and arrives at the presumed pathogenic mechanisms of the disease.

It has long been hypothesized that a deficit in the cerebral monoamine system (serotonin, dopamine and/or noradrenaline) is involved in the pathogenesis of depression and drugs that increase neurotransmission of these monoamines have been tested⁽¹³⁾.

It is also worth noting that reduced concentrations of 3-methoxy-4-hydroxyphenylglycol (a noradrenaline metabolite) are found in the liquor of patients with endogenous depression and high values are found in some patients with manic psychosis.

5-hydroxyindolacetic acid (5-HIAA), a deaminated metabolite of serotonin, is also present in lower than normal quantities in the liquor of depressed patients⁽¹⁴⁾.

Substance P probably also has an important role in causing depression⁽¹⁵⁾, because the blocking of substance P receptors has antidepressant effects.

Moreover, it has been observed that depressed patients and their first-degree relatives, as well as healthy individuals, develop a considerably depressed mood as a consequence of dietary deprivation of tryptophan, a precursor of monoamines.

Antidepressant drugs modify in different manners the activity of neurons, increasing monoamine levels and modulating the ion channels. Sodium channels are the molecular targets for antiepileptic drugs, which can also stabilize mood⁽¹⁶⁾.

Some of the most recent antidepressants act as selective inhibitors of serotonin reuptake and apparently performed their beneficial effects by increasing the quantity of functionally active serotonin in synapses (they also increase noradrenaline concentrations).

For these reasons, serotonin and its neural circuits also are currently believed to be involved in the genesis of depression. However, it must be noted that just slightly more than a decade ago it was widely believed that adrenaline depletion performed this role.

Besides, the reason why there is a delay of several weeks in depression improvement linked to the taking of various antidepressants is not explained by any of the neurochemical models⁽¹⁾.

Observations that the presence of a disorder of the hypothalamic-pituitary-adrenal axis might worsen the depressive state are suggestive⁽¹⁷⁾.

It has been known for several decades that parenteral administration of 1-2 mg of dexamethasone is unable to suppress the secretion of cortisol when the patient is affected by endogenous depression, while suppression occurs during remission.

Failure of suppression with dexamethasone has been attributed to neuron hyperactivity along the hypothalamic-pituitary axis and accordingly to an increase in the secretion of corticotropin releasing hormone (CRH), of ACTH and of glucocorticoids and induction of anxiety; moreover, it has been hypothesized that high levels of glucocorticoids prevent neurogenesis at the level of the mesial temporal lobe and might perhaps cause or facilitate the loss of hyppocampal neurons demonstrated in some studies on the brain of deceased depressed patients.

The idea that electroconvulsive therapy acts by increasing the levels of neurotrophic factors is at least in agreement with this view and with the hypothesis that a component of the remission of depression is in some way associated with the reconstitution of the normal neuronal architecture in the hyppocampal and hypothalamic regions⁽¹⁸⁾.

Although this is a highly speculative thesis, perhaps some of these changes might explain the delay in clinical improvement that can be observed after administration of antidepressants⁽²⁾.

There is strong evidence that depression involves alterations in many aspects of immunity, which contribute to the development or worsening of a number of medical disorders and can also have a role in the physiopathology of depressive symptoms.

This immune-mediated hypothesis is supported by indirect evidence of experimental and clinical studies of the effects of cytokines on behavior, which have shown that peripheral and central cytokines can cause depressive symptoms⁽¹⁹⁾.

### b) Anxiety

Studies on animals have linked acute anxiety with functional alterations of the locus coeruleus and of the septal and hyppocampal areas, i.e., the main noradrenergic nuclei, in addition to the role of the amygdala and to nuclei of the superior region of the encephalic trunk.

Presumably, some parts of the limbic system also are involved randomly in the production and perpetuation of anxiety and of the states related to it.

It should be noted that the locus coeruleus is involved in REM sleep and that drugs that suppress REM sleep, such as tricyclic antidepressants and monoamine oxidase inhibitors, reduce anxiety.

Other studies have linked to anxiety cerebral serotoninergic receptors other than the ones involved in depression.

Tests conducted by using positron emission tomography (PET) on subjects expecting to receive an electric shock have found an increase in activity at the level of the temporal lobes and of the insula, demonstrating that these regions are involved in the experience of acute anxiety.

Other investigations have demonstrated a role of the anterior part of the cingulum in evoking many of the vegetative characteristics (in particular an increase in heart rate), a state of excessive vigilance and anxiety. The discovery that a part, albeit a small one, of the hereditary characteristics of personality can be tied to polymorphism of the serotonin transporter gene is very interesting⁽²⁰⁾.

Another interesting alteration is that the levels of lactic acid are excessively high both at rest and after physical exercise and that the infusion of lactic acid can trigger panic attacks.

Hyperactivity of noradrenergic neurons (NE) and excessive release of NE from nervous terminals that occur in anxiety also cause a series of events at the level of postsynaptic NE receptors.

This leads to excessive signals at the level of the post-sympathetic beta adrenergic receptors both in second messengers and in subsequent messengers that mediate the autonomic symptoms associated with anxiety, including tachycardia, pupil dilation, tremor and sweating.

Indeed, the reactivity of the vegetative nervous system in these patients remains high and some stimuli (cold, pain, muscular effort) can cause disproportionate responses of heart rate, respiration, oxygen consumption and physical performance⁽²¹⁾.

In some patients urinary excretion of adrenaline is high; in others there is an increase in the excretion of noradrenaline and of its metabolites. During periods of intense anxiety, aldosterone excretion increases two-three times with respect to the norm.

As already mentioned, an increase in activity in the nervous pathways that use corticotropin releasing hormone CRH (from the amygdala to the hypothalamus, to the raphe nuclei, to the locus coeruleus and to other regions of the brain stem) induces anxiety; blocking this activity either pharmacologically or by destruction of the amygdala eliminates anxiety and causes the disappearance of fear-related behavior. [CRH is widespread in the encephalon. There is feedback on CRH and ACTH by means of receptors for glucocorticoids in the hypothalamus and in the anterior lobe of the hypophysis]. Serotonin and acetylcholine stimulate ACTH secretion, while catecholamines inhibit it⁽²²⁾.

Gamma-aminobutyric acid (GABA) is the main inhibitory neurotransmitter of the CNS of mammals. It has in fact been calculated that over 30% of all central synapses are of the GABAergic type.

There are three major types of post-synaptic GABA receptors: GABA-A, GABA-B, GABA-C.

GABA-A is a protein composed of five subunits; each subunit then has several subtypes. Subunit α contains the binding site for benzodiazepines and subunit β contains the binding site for GABA.

The benzodiazepine site regulates positively (agonist action) or negatively (inverse agonist action) the action of GABA on the receptor and requires the presence of subunit © to apply its effects.

Benzodiazepines enhance the activity of GABA, acting as positive allosteric modifiers, causing a conformational variation of the GABA-A protein complex, increasing receptor affinity for GABA. In turn, GABA causes a selective increase in permeability of the cell membrane to chlorine ions, inducing post-synaptic or presynaptic inhibition.

### Therapy

Due to the high degree of comorbidity of depression and of generalized anxiety, as well as of subtypes of anxiety disorder, the “maximum ambition” for a psychotropic drug is to combine antidepressant action with anxiolytic action.

Otherwise, patients treated with an antidepressant that is ineffective for concomitant states of anxiety will see their depression symptoms improve, but will continue to suffer due to the symptoms of anxiety. As an alternative, two agents must be administered simultaneously, one effective against depression and one effective against anxiety disorder⁽²¹⁾.

It should be stressed that meta-analyses of several extensive studies on the therapeutic effects of antidepressants have indicated that the clinical improvement that can be attributed to the drugs themselves occurs only in approximately half of the patients; the fact is noteworthy that in a further 25% of cases improvement can be ascribed to the placebo effect or to the natural course of the disease.

The remaining patients do not improve or have relapses while they are in therapy.

Tricyclic antidepressants comprise amitriptyline, imipramine, doxepine, clomipramine and strictly correlated drugs such as desipramine, nortriptyline and protriptyline.

The therapeutic effect of tricyclic drugs generally does not become apparent sooner than 2-4 weeks after the beginning of treatment. Common side effects: orthostatic hypotension, dry mouth, constipation, tachycardia, delay or inability to begin urination, tremor, sleepiness, risk of acute angle-closure glaucoma.

Currently most psychiatrists prefer to begin treatment with one of the functional agonists of serotonin (fluoxetine, sertraline, paroxetine, citralopram, etc.) or with one of the correlated drugs (for example venlafaxine and nefazodone). These drugs have a lower sedative and anticholinergic effect than tricyclic antidepressants and are not cardiotoxic.

However, in patients with anorexia, insomnia and intense anxiety, a more sedative drug such as amitriptyline is more useful.

If one of the drugs cited above, administered at full doses for 4-6 weeks, does not produce the desired effect, it is necessary to make an attempt with drugs that belong to other groups, for example MAOI (phenelzine, isocarboxazid, tranylcypromine). The most severe side effect of MAOI is the onset of hypertensive crises.

It has become common practice to add to the conventional antidepressant an anticonvulsant, in particular valproate or gabapentin, but also carbamazepine or phenytoin.

Few credible studies have assessed the value of this strategy, but these drugs can provide some additional benefits as “mood stabilizers”.

Treatment should be continued for 4-6 months, in association with a form of psychotherapy. Dosage should be reduced slowly over a period of a few weeks, since a rapid reduction can cause withdrawal symptoms (nausea, vomiting, illness and muscle pains).

If the depressive symptoms reoccur after suspension, the treatment must be resumed, generally until the dose that has proved itself effective is reached⁽²⁾.

Depression and subtypes of anxiety disorder are treated simultaneously with selective serotonin reuptake inhibitors (SSRI), and this approach often requires a single drug.

However, this is not always true for patients with major depressive disorder and a concomitant generalized anxiety disorder (GAD). In such cases, depression is often considered a priority in the hierarchy of symptoms, with the result that some patients can respond with a reduction of their general overall depression symptoms, but continue to have generalized anxiety symptoms instead of healing completely to a state of asymptomatic well-being.

In any case, considering the trend that shows that some antidepressants are also anxiolytics, today more than before it may be possible, in the common situation in which patients have a major depressive disorder and a GAD, to eliminate both the symptoms of depressed mood and the symptoms of anxiety.

This therapeutic result would lead patients with depression and concomitant generalized anxiety to a state of well-being without residual symptoms of depression or anxiety⁽²¹⁾.

As regards panic attack therapy, some drugs, particularly benzodiazepines, are sometimes very effective in suppressing them and in inducing a sense of well-being, but these drugs, especially if taken for the first time, commonly cause fatigue; with prolonged use they can cause addiction; they do not protect against relapses when treatment is interrupted, even after prolonged administration (6 to 12 months).

Buspirone, a 5HT₂ specific serotoninergic agonist, has been proposed for the treatment of anxiety and as a replacement of benzodiazepines, but its effectiveness is limited.

It is important to stress that during the initial weeks of administration of antidepressants the underlying symptoms of anxiety may worsen and usually an anxiolytic is necessary until the antidepressants begin their effect.

Propranolol, or an adrenergic blocker with prolonged release, reduces greatly many of the accompanying symptoms of anxiety and is useful to many patients, but its effects on the other symptoms are uncertain⁽²⁾.

Tricyclic antidepressants and SSRI drugs, which hypothetically increase the concentrations of serotonin at the level of the nervous system, are effective to a certain extent in preventing panic attacks and agoraphobia, but the beginning of their effect is delayed by weeks: they are useful for anxiety symptoms that persist for several months. The doses are similar to those used for depression.

Although selective serotonin reuptake inhibitors (SSRI) produce clinical therapeutic effects on depression and anxiety, increasing serotoninergic neurotransmission, little is known regarding the potential contribution of the 5-HT(6) receptor in the treatment of mood disorders.

Available data suggest that the agonist receptor 5-HT(6) can be a new class of potential antidepressant and anxiolytic compounds and might be an advantage with respect to currently available treatments, such as a rapid beginning of anxiolytic action⁽²³⁾.

Benzodiazepines increase the effect of the gamma-aminobutyric acid (GABA) neurotransmitter, thus increasing its sedative, hypnotic, anxiolytic, anticonvulsant, anesthetic and myorelaxant properties.

They are usually safe and effective in short-term treatments, whereas long-term use is more problematic due to the development of tolerance, the risk of physical and psychic addiction with consequent withdrawal syndrome.

Sudden interruption of treatment can cause insomnia, tremor, anxiety and panic attacks, anorexia, tachycardia, hypertension, dysphoria, depression and suicidal ideation.

### SUMMARY

In view of these data of physiopathology and biochemistry of anxiety and depression, the present disclosure provides a compound that is effective on the various syndromic clinical pictures outlined above.

A second object is to provide a compound that has characteristics of harmlessness.

A third object is to provide a compound that has rapid action.

A fourth object is to provide a compound that has no side effects.

A fifth object is to provide a compound that does not have habituation, tolerance and addiction phenomena and therefore without problems of onset of withdrawal symptoms and of rebound effect upon treatment suspension.

A sixth object is to provide a compound that is effective both on anxiety and on depression.

A seventh object is to provide a broad spectrum compound with a multifactor mechanism of action on the biochemical components of anxious and depressive syndromes.

A further object is to provide a compound that is effective on the various syndromic clinical pictures outlined above.

The proposed objects, which can be deduced from the description and from the accompanying five figures, are achieved by a compound particularly for treating depression and anxiety, characterized in that it comprises the combination of at least 11-keto-beta-boswellic acid (KBA) and acetyl-11-keto-beta-boswellic acid (AKBA).

Furthermore, limonene and 1,8-cineol can be associated with these acids.

### BRIEF DESCRIPTION OF THE DRAWINGS

FIG. 1 shows the structural formula of incensol acetate;

FIG. 2 shows the structural formulas of the two boswellic acids according to a first embodiment of the present disclosure;

FIG. 3 is a table showing test data wherein the products were formulated with 80% ethyl alcohol, water as needed;

FIGS. 4 and 5 are tables showing the results and summary of the tests from FIG. 3; and

FIGS. 6-8 are histograms of the tests conducted.

### DETAILED DESCRIPTION OF COMPONENTS AND THEIR ACTION

### A. Action of Boswellic Acids: [11-keto-beta-boswellic acid (KBA) and acetyl-11-keto-beta-boswellic acid (AKBA)]

Frankincense or olibanum is a resin produced by Boswellia plants of the Burseraceae family and is known since antiquity for its curative properties.

The Boswellia genus of the Burseraceae family is divided into about fifteen species. It is original to the Persian Gulf in the Indian Ocean and is cultivated in several countries, such as southern Arabia, Somalia, Ethiopia, Eritrea, Sudan and Kenya.

Boswellia serrata is cultivated in India.

The best resin is harvested in autumn, following incisions made in the summer, and produces what is known as a white incense by contrast with red incense, which is gathered in spring following incisions during the winter.

The essential incense oil, which is composed mainly of pentacyclic triterpenes derived from boswellic acid, is extracted with various methods from this oily, resinous, fragrant, transparent and yellow-brownish gum. The following are reported:

actions supported by clinical trials: anti-inflammatory, anti-arthritic, analgesic, antitumor, anti-asthmatic and aromatic;

actions supported by research on animals and in vitro: anti-inflammatory, analgesic, immune modulating, anticarcinogenic, antihyperlipidemic, antifungal, antipyretic, aromatic;

historical or theoretical actions that lack appropriate evidence: anti-catarrhal, mucolytic, expectorant; uterine stimulant, emmenagogue, diuretic, antidepressant, emulgent, emollient, laxative, in amenorrhea, against syphilis, against urinary infections, anticancer.

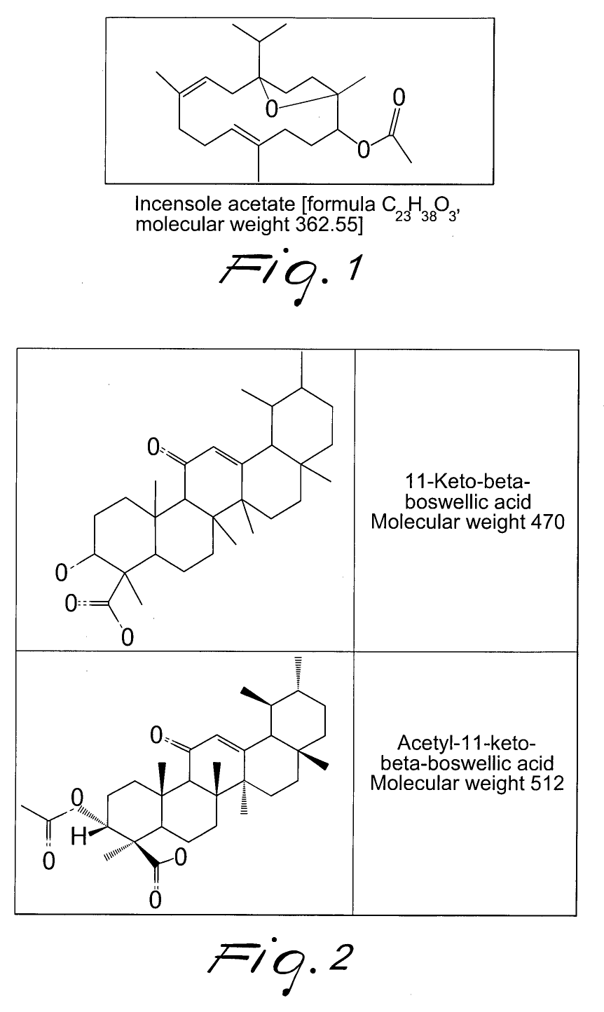

Recently, incensol acetate (see FIG. 1) has been extracted: a diterpene incense component that has an anti-anxiety and antidepressant psychoactivity, with a mechanism of activation of the TRP3 channels in the brain⁽²⁴⁾.

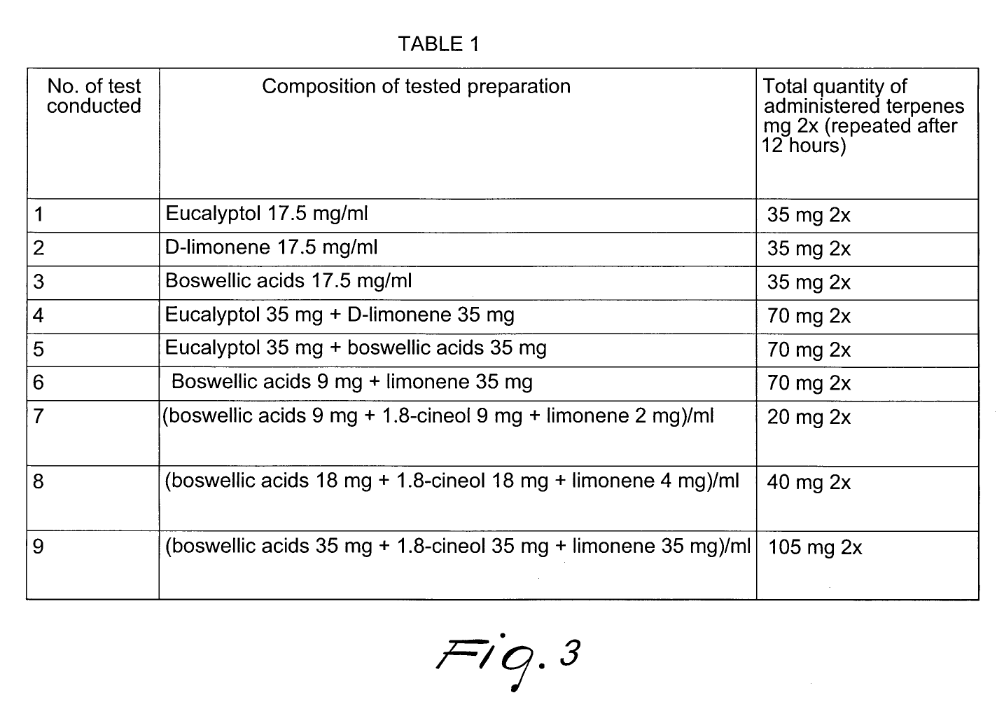

FIG. 2 shows the structural formulas of the two boswellic acids to which the present disclosure mainly relates.

### Literature Data

In vivo studies demonstrate that boswellic acids are safe enough and exhibit no genotoxicity up to a dose of 1000 mg/kg in rats⁽²⁵⁾ and no organic lesions beyond 5000 mg/kg⁽²⁶⁾.

The content of boswellic acids in Boswellia serrata is quite different⁽²⁷⁾:

Alpha-beta-boswellic acid 8.68-16.1 mg/g
Beta-boswellic acid 53.5-246.9 mg/g
3-acetyl-beta-boswellic acid 38.4-192.9 mg/g
11-keto-beta-boswellic acid 4.48-5.81 mg/g
11-keto-beta-acetyl-boswellic acid 32.7-44.2 mg/g

### Metabolism of Boswellic Acids In Vitro and In Vivo

After oral administration to young volunteers of 333 mg of Boswellia serrata extracts, elimination half-life is approximately 6 hours and this suggests that the drug must be administered at this rate. The steady-state of plasma concentration is reached after approximately 30 hours. At this dose the preparation is well tolerated and has no side effects⁽²⁸⁾.

Pharmacokinetics studies have demonstrated the poor bioavailability of boswellic acids, particularly the two most powerful ones: 11-keto-beta-boswellic acid and 3-acetyl-11-keto-beta-boswellic acid, and this is due essentially both to modest oral absorption⁽²⁹⁾ and to blocking in passing through the blood-brain barrier: for 11-keto-beta-boswellic acid (KBA) and acetyl-11-keto-beta-boswellic acid (AKBA), the brain/plasma ratio is respectively approximately 0.51 and 0.81, despite their lipophilic nature. It is believed that this is due to their interaction with transporters, particularly with P-glycoproteins (PGP), both at the intestinal and cerebral level⁽³⁰⁾ or rather because KBA and AKBA modulates the activity of OATP1B3 (organic anion transporter protein 1B3) and of MRP2 (multidrug resistance-associated protein 2)⁽³¹⁾. To increase oral absorption, they have been complexed with phosphatidylcholine⁽³²⁾. Oral absorption is increased considerably by a fat-rich diet⁽³³⁾.

Therefore, the privileged administration pathway of terpenes and of boswellic acids in particular is inhalation, or more precisely “the olfactory terpene pathway”.

### The “Olfactory Terpene Pathway” to the Central Nervous System

A glycoprotein has been identified that is specific for nasal tissues, which is indeed termed “olfactory binding protein” and is capable of transporting small lyophilic molecules, such as terpenes, and is recognized specifically by receptors expressed on the surface of olfactory cells.

It is reasonable to hypothesize that, once the receptors of the olfactory cells have been saturated, during the inhalation of terpenes the olfactory binding proteins transport the terpenes and pass rapidly into the blood in a higher proportion than the terpenes bound to Pgp and therefore reach the right heart in a few seconds and, after pulmonary circulation, are introduced in general circulation, so that in less than one minute they reach directly the brain and the spinal cord with cerebral and spinal arterial blood, without undergoing liver metabolism at their first passage through circulation. Terpenes pass beyond the blood-brain barrier and with choroidal filtration pass into the cerebrospinal liquor (CSL).

CSL can be considered an excellent vehicle for terpenes: indeed, since they have a higher concentration therein with respect to the liquid of the extracellular compartment, an inverse motion of terpenes between CSL, extracellular liquid and brain cells is established.

In a test animal, the elimination of CSL occurs predominantly by means of the olfactory nerve (open connections have been demonstrated between the perineural space of the fibers of the olfactory nerve and initial lymphatic vessels in nasal mucous membrane)^{(33,34)}; to a much smaller extent: through the perineural spaces of the optic nerve, which drains behind the eyeball in orbital tissue; through the vestibulo-cochlear nerve, which drains into the perilymphatic spaces of the inner ear; through the membrane of the oval window in lymphatic vessels of the middle ear. It is therefore logical to deduce that the elimination of CSL and therefore of terpenes can occur also through the perineural spaces of the other cranial nerves.

To conclude, the olfactory pathway ensures the traffic of terpenes from the nose to the central nervous system and vice versa, with an absolutely simple manner, i.e., the inhalation pathway. All this opens new prospects in the treatment of diseases of the brain and of cranial nerves:

of meninges, such as microbial, bacterial and viral infections;

of the inner ear, such as Ménière's disease, vestibular neuronitis, herpes zoster oticus, vertigo, drugs-induced ototoxicity;

of the middle ear, such as otitides and mastoidites;

of the optic nerve and of the ocular orbit, such as retrobulbar neuritis, toxic amblyopia, optical atrophy, orbital cellulite, thrombosis of the cavernous sinus;

of the olfactory nerve and of the ethmoid, as in disorders of the sense of smell and in ethmoiditises;

probably also of other cranial notes, such as in paralysis of the facial nerve, in trigeminal neuralgia, in swallowing disorders.

Boswellic acids constitute the main pharmacological principles of incense, but their targets and underlying modes of molecular action are still unclear; the ones that are known so far are summarized below.

### Anti-Inflammatory

Boswellic acids inhibit: the activation of NF-kB⁽³⁵⁾ and STAT proteins⁽³⁶⁾ with down-regulation of TNF-α and reduction of inflammatory cytokines IL-1, IL-2, IL-4, IL-6 and IFN-γ; of 5-lipoxygenase, with consequent reduced production of leukotrienes; selectively, cyclooxygenase 1 (COX-1)⁽³⁷⁾, considered the molecular base of the anti-inflammatory activity of boswellic acids⁽³⁸⁾; the forming of oxygen and protease radicals, such as cathepsin G and elastase⁽³⁹⁾; the expression of TNF-α induced by matrix metalloproteinases (MMPs) of matrix and the activity of MMP-3, MMP-10 and MMP-12⁽⁴⁰⁾; lipopolysaccharide activity⁽⁴¹⁾; inflammatory angiogenesis⁽⁴²⁾.

Therefore, there is no surprise regarding the positive effects of boswellic acids in some chronic inflammatory diseases, such as rheumatoid arthritis, bronchial asthma, osteoarthritis, ulcerous colitis, Crohn's disease⁽⁴³⁾ and immune-based diseases such as psoriasis⁽⁴⁴⁾ and pulmonary fibrosis induced by bleomycin⁽⁴⁵⁾; in experimental autoimmune encephalomyelitis⁽⁴⁶⁾.

### Antimicrobial

Boswellic acids act on strains of Staphylococcus mutans and Actinomyces viscosus, Enterococcus faecalis and faecium, Streptococcus sanguis, Prevotella intermedia, Porphyromonas gingivalis⁽⁴⁷⁾ and Staphylococcus Staphylococcus aureus and epidermidis probably by destruction of the structure of the microbial membrane⁽⁴⁸⁾.

### Antitumor

Recent preclinical studies have demonstrated that boswellic acids have an anticancer potential against various tumors. They inhibit the expression of: transcription factors of specific proteins (Sp) and of numerous genes regulated by pro-oncogene Sp in multiple cancer cell lines⁽⁴⁹⁾; the expression of transcription factors such as Nf-kB and STAT3⁽⁵⁰⁾; of COX-2, MMP-9, CXCR4, VEGF⁽⁵¹⁾ and of topoisomerases I and II⁽⁵²⁾.

Encouraging results have been observed in prostate tumor⁽⁵³⁾, in cervical carcinoma⁽⁵⁴⁾, in colorectal cancer⁽⁵⁵⁾, in Ehrlich's solid experimental tumor⁽⁵⁶⁾.

Boswellic acids inhibit the growth of glioma⁽⁵⁷⁾, reducing peritumoral edema⁽⁵⁸⁾, and have a cytotoxic action on the cells of meningioma⁽⁵⁹⁾, so that extracts of Boswellia serrata were designated in 2002 by the European Medicines Agency as orphan drugs.

### Neurotrophic and Neuroprotective

Boswellic acids protect nerve cells against excitotoxic insults arising from oxidative damage and from excessive stimulation of glutammatergic receptors⁽⁶⁰⁾, preventing damage of axonal integrity, and have the capacity to produce axonal excretions and ramifications and to influence the dynamics of tubulin polymerization: this also explains the use, in traditional ayurvedic medicine, of boswellic acids to prevent amnesia⁽⁶¹⁾.

### Anti-Nociceptive

Boswellic acids enhance the action of NSAID drugs for lipoxygenase inhibition⁽⁶²⁾. It is believed that boswellic acids have the same anti-nociceptive mechanism of action, for example on bowel and pelvic pain, of other triterpenes, such as oleanolic acid, which involves endogenous opioid receptors, nitric oxide and opening of K(ATP) channels⁽⁶³⁾ and vanilloid receptors (TRPV1), as for the triterpenes alpha- and beta-amyrin⁽⁶⁴⁾.

### Antidepressant

It is probable that boswellic acids have an antidepressant action with the same mechanism of action of other triterpenes which induce, in rat brain, a significant reduction in the level of serum corticosterone and an increase in 5-HT, NE, DA and of their metabolites 5-HIAA, MHPG⁽⁶⁵⁾.

### B. Actions of Limonene

Limonene is one of the most common terpenes in nature, with a lemon-like sweet scent, the main constituent of many citrus fruit oils, such as orange, lemon, mandarin, grapefruit. By virtue of its pleasant scent of citrus fruits, d-limonene is used widely as an additive in perfumes, soaps, foods, beverages and chewing gums.

D-limonene can also be used as an inert ingredient in pesticides and as a natural substitute of petroleum-based solvents in paints and in cleaning products.

It has low toxicity. Orally, d-limonene is absorbed rapidly in the intestinal tract both in man and in animals. In the rat, oral bioavailability is 43.0%⁽⁶⁶⁾.

In healthy volunteers exposed to inhalation of d-limonene for 2 hours during a 50 W workload, the corresponding pulmonary uptake was 70% of the administered quantity. D-limonene is metabolized promptly. A long hematic t1/2 was observed in the slow stage of elimination, and this indicates an accumulation in adipose tissue. A decrease in vital capacity was observed after exposure to d-limonene at high concentrations. No irritative symptoms or symptoms correlated with the CNS⁽⁶⁷⁾.

The thresholds of sensory irritation in mouse and man are rather close. R-(+)-limonene has irritating effects on the respiratory system at a concentration below 1599 ppm and S-(−)-limonene, lower than 2421 ppm. Both enantiomers induce slight bronchoconstriction below 1000 ppm⁽⁶⁸⁾.

### D-Limonene: Literature Data

D-limonene is attributed the following activities:

Skin Penetration Enhancer

Used at different concentrations as a permeation promoter of drugs administered transdermally⁽⁶⁹⁾.

Tissue Lipophilic

It tends to accumulate in cells with a rich content of fat, such as subcutaneous adipose tissue, the mammary gland, type II pneumocytes producing pulmonary surfactant, nerve cells; it is an excellent solvent of cholesterol and has antihyperlipidemic effects⁽⁷⁰⁻⁷⁴⁾.

Anti-Inflammatory

It inhibits in macrophages the production, induced by lipopolysaccharide (LPS), of NO and prostaglandin E₂ and reduces in a dose-dependent manner the expression of iNOS, COX-2, TNF-α, IL-β, and IL-6⁽⁷⁵⁾.

Antiproliferative and Chemopreventive

It induces apoptosis and chemoprevention by means of the inhibition of inflammation, of the oxidative stress and of Ras-signal⁽⁷⁶⁾. It is neither genotoxic nor carcinogenic but has a chemopreventive activity against many types of tumor, capable of protecting DNA against free radicals and of inhibiting the growth of tumor cells, so that orange juice can be a candidate as a nutraceutical⁽⁷⁷⁾.

Gastroprotective

Increases the production of gastric mucus and maintain base levels of PGE₂ after challenge with harmful agents⁽⁷⁸⁾.

Vasomotor

Inhaled by healthy volunteers, it induced an increase in blood pressure⁽⁷⁹⁾; vice versa, in hypertensive subjects d-limonene is effective in reducing systolic pressure (not diastolic pressure) and the activity of the sympathetic nervous system (lower heart rate variability)⁽⁸⁰⁾. Inhaling (−)-limonene causes an increase in systolic blood pressure but has no effect on psychological parameters⁽⁷⁹⁾; moreover, it has no relaxing action on the smooth muscles of the ileum in test animals^{(81, 82)}.

anticholinesterase, which can have prospects in the treatment of Alzheimer's disease⁽⁸³⁻⁸⁵⁾.

anti-nociceptive, analgesic in bowel pain, aspecific, due to inhibition of synthesis or release of inflammation mediators, not in relation to the stimulation of opioid receptors⁽⁸⁶⁾.

on attention, anxiolytic and antistress

Inhaled by healthy volunteers, it induced greater attention but also restlessness, while inhalation of (−)-limonene has no effects on psychological parameters⁽⁷⁹⁾.

It has antianxiety and antistress activity⁽⁸⁷⁾ because:

it induces an increase in the GABA content in the brain. GABA, one of the most widespread neurotransmitters in the brain and dominant neurotransmitter in the hypothalamus, has a vital role in the antistress process: it induces hyperpolarization, opening the CF channels; it regulates the activity of GABAergic neurons and of other types of neuron; for example, it has been reported that GABAergic neurons regulate the activity of 5-HTergic neurons in the dorsal raphe nucleus (DR), which is projected to many brain regions, such as the hypothalamus, hippocampus and amygdala. This explains why the administration of limonene reduces the concentration of 5-HT and of glutamate in the encephalon (which is the main excitatory neurotransmitter thereof), while it increases its GABA content⁽⁸⁸⁾.

it regulates the activity of the hypothalamic-pituitary-adrenal axis (HPA). The major components of the stress system are in fact the hypothalamic-pituitary-adrenal loop, and the locus coeruleus-noradrenaline-autonomic system path. In conditions of acute stress, the stress factors cause a release of CRH (corticotropin-releasing factor) from the paraventricular neurons in the hypothalamus and therefore activate the release of ACTH from the hypophysis. The release of corticosterone is controlled by ACTH. The administration of agonists of some GABA receptors inhibits the production of corticosterone in response to the stress, while the increase in the release of CRH increases its production. Therefore, limonene, which induces a reduction in 5-HT, one of the factors that facilitate the activation of the HPA axis, can contribute to reducing the responds of the HPA axis to stress⁽⁸⁸⁾.

Antidepressant

Limonene accelerates significantly the metabolic turnover of DA in the hippocampus and of 5-HT in the prefrontal cortex and in the striatum, and this suggests that has antidepressant-like effects by suppressing DA activity linked to higher activity of 5-HTergic neurons⁽⁸⁹⁾.

Inhaling limonene reduces significantly the immobility time and enhances the effect of imipramine, a tricyclic antidepressant, in rats with the swimming test, probably influencing receptor-mediated GABA_{A} response⁽⁹⁰⁾.

Inhaling limonene can be a stimulant for the CNS, since it extends the latency time of sleep and shortens sleep⁽⁹¹⁾.

Anticonvulsant

This is probably linked to a mechanism of inhibition of GABA uptake and of the bond with GABA in a dose-dependent manner⁽⁹²⁾.

Presumably, the two enantiomers, d-limonene and s-limonene, have the same mechanism of action, since s-limonene also attenuates the effect of an antagonist of the receptor site of benzodiazepine on GABA_{A}, flumazenil.

### C. Actions of 1,8-cineol

Eucalyptol, or 1,8-cineol, is a natural substance that at ambient temperature is a colorless oily liquid with a camphor-like smell and a pungent taste. Chemically it is a heterocyclic monoterpene.

It is prepared synthetically or extracted by fractional distillation of essential oil obtained from eucalyptus leaves. It is used in perfumery or medicine as an antiseptic, balsamic, anti-catarrhal product. Acute toxicity in mouse: oral dose 2480 mg/kg.

1,8-cineol is absorbed rapidly with respiration and can be detected in blood 5 minutes after inhaling. Elimination is slightly different in the two sexes, from 2.5 to 10 times slower in females, probably due to the higher ratio between body fat and body weight⁽⁹³⁾.

It diffuses more rapidly after inhaling than after oral administration and through the skin. Its presence in blood was detected 5 minutes after inhaling with a concentration maximum within 18 minutes⁽⁹⁴⁻⁹⁶⁾.

The following activities are attributed to 1,8-cineol:

Anti-Inflammatory

Inhibits the production of TNFα, cytokines (IL-1β, IL-4, IL-5, IL-6, IL-8) and prostaglandins^{(97, 98)}.

In human monocytes stimulated by LPS, reduces the expression of Egr-1 (early growth response factor-1), a transcription factor of many important inflammation genes, but is has no effects on the expression of NF-kB, a key transcription factor of inflammation genes⁽⁹⁹⁾.

Inhibits the LPS-induced production of NO⁽¹⁰⁰⁾.

Reduces myeloperoxidase activity and restores glutathione⁽¹⁰¹⁾.

Therefore, it is useful in the treatment of rhinitis, sinusitis, bronchitis, COPD, asthma and coughing both for its anti-inflammatory activity and for its secretolytic action, bronchial myorelaxant action and action of improvement of mucociliary clearance⁽¹⁰²⁻¹⁰⁵⁾.

Gastroprotective

It is gastroprotective against damage induced by ethanol in the rat, with reduced production of gastric juice and of total acidity, attributed to antioxidant activity and lipoxygenase inhibition activity⁽¹⁰⁶⁾.

Cardiovascular

Increases blood circulation with skin hyperemia after local application and increases cerebral blood flow after prolonged inhaling or after oral administration^{(107, 108)}.

Reduces the heart rate by parasympathetic-dependent action and hypotensive effects by direct action on smooth vascular muscles rather than a loss of sympathetic tone⁽¹⁰⁹⁾.

Depresses cardiac isometric contraction by blocking calcium channels⁽¹¹⁰⁾.

anti-acetylcholinesterase and anti-butyrylcholinesterase action.

With these characteristics, it has favorable prospects for treating Alzheimer's disease^{(111, 113)}.

Skin Penetration Enhancer

Eucalyptol, like limonene, increases percutaneous penetration of drugs, for example antipsychotic drugs^{(114, 115)}.

Analgesic and anti-nociceptive action

It has a manifest bowel analgesic action by cutaneous topic application⁽¹¹⁶⁾ and a modest local anesthetic action⁽¹¹⁷⁾.

The anti-nociceptive action is comparable to morphine in the rat⁽¹¹⁸⁾ but with a non-opioid mechanism⁽¹¹⁹⁾, since an oral dose with a range between 100 and 400 mg/kilograms is not canceled out by pretreatment with naloxone, an antagonist of μ-opioid receptors.

The anti-nociceptive activity is performed presumably by desensitizing of TRPV3 (transient receptor potential vanilloid-3), which is a heat-sensitive ion channel that is expressed in skin keratocytes and in a variety of nerve cells, is activated by heat and by mono terpenes, such as 1,8-cineol. Indeed, prolonged exposure to 1,8-cineol induces agonist-specific desensitizazion of endogenous TRPV3 with allosteric mechanism⁽¹²⁰⁾.

Antianxiety and Antidepressant

The antianxiety and antidepressant activity of 1,4-cineol was demonstrated by Gomes P B et al.⁽¹²¹⁾ with various tests, such as elevated plus maze (EPM), hole board, open field, pentobarbital sleeping time, forced swimming, tail suspension and rotarod tests. 1,8-cineol was administered orally to the mouse at a dose of 100, 200 and 400 mg/kg, while diazepam (1 or 2 mg/kg) and imipramine (10 or 30 mg/kg) were used as reference drugs. 1,8-cineol at 200 and 400 mg/kg induced a significant increase in the immobility time and a reduced latency of pentobarbital sleep. Cineol exhibited no effects on motor coordination in the rotarod test.

The results suggest that 1,8-cineol has an anxiolytic action, with possible general depression of CNS^{(119, 121)}.

The anti-anxiety action is confirmed by other authors⁽¹²²⁾ although it is not unanimous (non-significant anti-conflict effect in the Geller test)⁽¹²³⁾, but essential rosemary oil, which has among its main components 1,8-cineol, has an antidepressant effect that is probably mediated by interaction with the monoaminergic system⁽¹²⁴⁾ and essential lavender oil, which contains 1,8-cineol at the concentration of 3-5%, has an anxiolytic, anticonvulsant and sedative activity for enhancement of GABA_{A}-receptor response⁽¹²⁵⁾.

In summary:

### 1.—Actions of Boswellic Acids (KBA and AKBA)

The anti-depressant and antianxiety activity of boswellic acids is not known and is demonstrated for the first time with this disclosure. The mechanism of action can only be hypothesized and will be the subject of subsequent research.

However, it is likely that some activities of boswellic acids cooperate, in particular:

anti-inflammatory and analgesic activities, because they inhibit:

the activation of NF-kB and of STAT proteins and consequent down-regulation of TNF-α and reduction of pro-inflammatory cytokines IL-1, IL-2, IL-4, IL-6 and of IFN-γ;

5-lipoxygenase, with consequent reduced production of leukotrienes;

selectively, COX-1, with down-regulation of the expression of COX-2;

the forming of oxygen radicals.

Since boswellic acids are powerful inhibitors of inflammation and immune modulators and considering that experimental studies highlight the effect of cytokines on behavior, the result is that boswellic acids have a non-secondary influence in the antidepressant action.

Neurotrophic and neuroprotective, specifically against excitotoxic damage induced by depression or by drugs or by cerebrovascular disorders or by neurological diseases or by hormone imbalances (see depression in women) which can cause depression as in a vicious circle.

The idea that boswellic acids act by raising the levels of neurotrophic factors with reconstitution of normal neuronal architecture especially in the hippocampae and hypothalamus regions (as hypothesized by Chan for electroconvulsive therapy⁽¹²⁹⁾) is in agreement with the observation of the durable remission of depression after therapy with the preparation according to the present disclosure.

Moreover, it can be hypothesized that boswellic acids, due to their structural affinity with cortisones, can contrast the toxic action of high levels of glucocorticoids, which, as reported above, in some studies on deceased depressed patients have demonstrated that they inhibit neurogenesis and on the contrary determine or facilitate the loss of hippocampal neurons.
Anti-nociceptive, enhancing the action of NSAID drugs for inhibiting lipoxygenase.

It is probable that boswellic acids have the same anti-nociceptive mechanism of action, for example on bowel and pelvic pain, as other triterpenes, such as oleanolic acid, which involves endogenous opioid receptors, nitric oxide and opening of the K(ATP) channels and vanilloid receptors (TRPV1), like for alpha- and beta-amyrin triterpenes.

Comparative analysis of the properties of limonene, 1,8-cineol and boswellic acids suggests that they can have a synergistic anti-anxiety and antidepressant action, since limonene acts specifically on GABAergic transmission and on the hypothalamic-pituitary-adrenal axis; that 1,8-cineol interacts on the monoaminergic and anti-nociceptive systems and on brain perfusion; that boswellic acids also have a marked anti-inflammatory, neurotrophic and neuroprotective activity.

### 2. Actions of Limonene

Limonene has an anxiolytic and antidepressant action because:
it induces an increase in GABA content, which can help to reduce depression and regularize the sleep-wake rhythm in OSAS, reducing its depression;
it reduces the concentration of 5-HT and glutamate in the encephalon;
it accelerates significantly the metabolic turnover of DA in the hippocampus and of 5-HT in the prefrontal cortex and the striatum;
it influences the regulation of the activity of the hypothalamic-pituitary-adrenal axis, inhibiting the production of corticosterone in response to stress;
it has an antiinflammatory action (inhibits production of NO and prostaglandin E₂, reduces the expression of iNOS, COX-2, TNF-α, IL-β, and IL-6);
it has an anti-nociceptive action with the same antiinflammatory action.

### 3. Actions of 1,8-Cineol

Eucalyptol has an anti-anxiety and antidepressant activity because:
it interacts with the monoaminergic system;
it reduces the latency of sleep induced by pentobarbital, without effects on motor coordination;
it has an analgesic and anti-nociceptive activity via an allosteric mechanism, with agonist-specific desensitization of TRPV3;
it has an antiinflammatory activity;
it inhibits the production of TNFα, cytokines (IL-1β, IL-4, IL-5, IL-6, IL-8), and NO induced by LPS;
it inhibits lipoxygenase and prostaglanding production;
it reduces myeloperoxidase activity and restores glutathione;
it reduces the expression of Egr-1 (early growth response factor-1), a transcription factor of many important inflammation genes;
it increases brain blood flow by direct action on vascular smooth muscles rather than by drop in sympathetic tone.

### A. Experimental Study on Mice of the Antidepressant Action of the Terpenes According to the Investigation, Individually and in Association

### Materials and Method

The following materials were used:

1. Boswellia serrata AKBAMAX batch BSAK-170/1109/B-25 in powder, having total boswellic acids titer of 43.1%; 3-O-acetyl-11-keto-boswellic acid 10.2%; acetyl beta-boswellic acids 25.4%, acetyl-alfa-boswellic acids 7.5%. Extraction by the company MEG and HPLC-MS analysis of the hydroalcoholic extract used in the tests, containing 11-β-keto-boswellic acid 2.9 mg/ml and acetyl-11-β-keto-boswellic acid 14.6 mg/ml; total boswellic acids 17.5 mg/ml.

2. Eucalyptol Ph. Eur.—N.F. CAS no. 470-82-6; EINECS/ELINCS No. 207-431-5 [supplied by ACEF S.P.A. (29017 Fiorenzuola D'Arda (PC), Via Umbria 8/14] solution containing 2.00 g in 10 ml of 96% ethanol.

3. (R)-(+)-limonene 97% 183164-5 ml Sigma Aldrich, Lot 1447241V Pcode 101079356 601-029-00-7 CAS 5989-27-5 C10H16.

4. For each test, CD-1® (ICR) Specific Pathogen Free NAF mice (16/18 g upon arrival) were used, originating from the supplier facility Charles River Laboratories, Calco, Italy; 5 mice for the control group, 5 mice for the reference test with imipramine, 5 mice for the substance tested in the first 4 tests and then 10 mice for all the other tests. The choice of CD1 mice was determined by the fact that it is considered to be the animal model suitable for performing the swimming test for the evaluation of substances having an antidepressant action.

5. For terpene vaporization, “Elettromatt Turispharma” electric vaporizers were used (PCT/EP 97/01897, inventors Bevilacqua M, Zaccagna C A), heated to a constant temperature of 90° C.

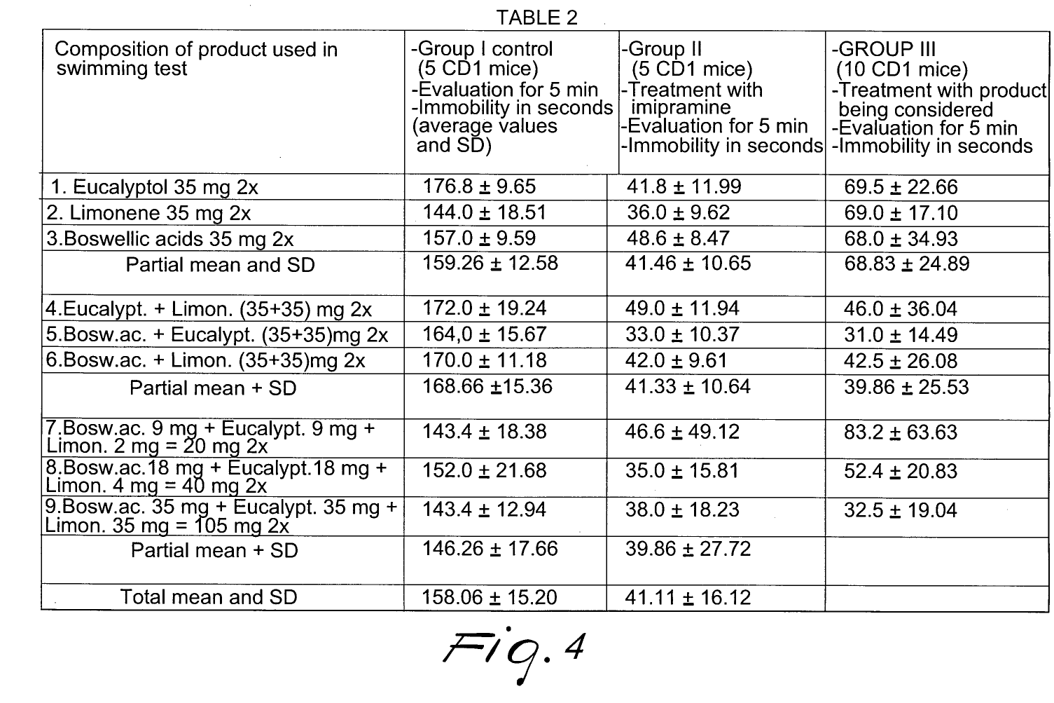

The products were formulated with 80% ethyl alcohol, water as needed and are listed in Table 1 of FIG. 3.

The animals were subjected to a period of acclimatization of 7 days before beginning the experimental procedures.

Each animal was identified by cutting the tip of the phalanxes of the anterior and posterior right paw, in accordance with Standard Operating Procedures.

The animals were stabled according to the indications provided by applicable Standard Operating Procedures.

The diet was of the standard type for maintenance of mice. Potable water was filtered and sterilized with appropriate filters. Access to food and drinking were ad libitum. Analyses of administered food were produced by the supplier. Drinking water was analyzed according to applicable Standard Operating Procedures to prevent any contaminants from being present so as to interfere with the study in progress.

The three groups of mice were placed in three transparent plastic boxes with the size of 150 cm×75 cm and a volume di 843,750 ml³ at a controlled temperature (19.0-21.0° C.), in air prefiltered with an HEPA absolute filter without flow, humidity (40-50%) and darkness/light cycle (12 hours/12 hours).

The same dropper was always used: 1 ml=42 drops.

The reference substance was imipramine chloride hydrate (Sigma 17379-5G; batch 011M0094V; 99% crystalline; solubility 50 mg/ml), injected intraperitoneally (IP).

The control group received the carrier (physiological solution) by IP pathway.

The tests with the substances being considered (boswellic acids, 1,8-cineol, limonene), on their own, in association and at various concentrations, were performed blind (with imaginary names), randomized (in order of execution, with respect to Table 1) so that the results were mutually unconnected and entirely unpredictable:

conducted test no. 1=no. 8 of Table 1
conducted test no. 2=no. 9 of Table 1
conducted test no. 3=no. 1 of Table 1
conducted test no. 4=no. 6 of Table 1
conducted test no. 5=no. 3 of Table 1
conducted test no. 6=no. 7 of Table 1
conducted test no. 7=no. 1 of Table 1
conducted test no. 8=no. 5 of Table 1
conducted test no. 9=no. 4 of Table 1

### Table 1 is FIG. 3.

### Administration Paths

The administration path of the substances being considered was intraperitoneal for imipramine and the pulmonary path was used for the terpene substances being considered, always vaporized with the same electric vaporizers.

### Calculation of Vaporized Doses

The first test (test no. 8, see composition in Table 1) was performed assuming that a terpene dose equivalent to the injected dose of imipramine was vaporized.

### Specifically:

Reference test substance: imipramine 30 mg/Kg/ip=30.000 mcg/Kg/ip, equal to 0.6 mg per animal, administered 30 minutes before the test.

Assessments of respiratory physiology for CD1 mice with an average body weight of 20 grams and a Tidal volume (Vₜ) of 0.2 ml with a breathing rate (Fr) of 163 acts/min:

Total dose to be injected into the 5 mice: 30 mcg×20×5=3000 mcg=3 mg.

Dose of active ingredient to be made to inhale: 3 mg=3000 mcg.

Ventilation/minute (=V_{T}×Fr) of the 5 mice: 0.20 ml×163×5=163 ml/min. Ventilation/24 h=163×60×24=234,720 ml.

Volume of box: 150 cm×75 cm×75 cm=843,750 ml³.

Volume of box in excess of ventilation volume/24 h of the mice battery: 843,750−234,720=609,030 ml.

Theoretical dose to be vaporizyed in the box volume in excess of mice ventilation over 24 h: 3000:234,720=x:609,030; x=7,784 mcg.

Theoretical total dose to be vaporized in box: 3000+7,784=10,784 mcg.

Real total vaporized dose, calculating that absorption through inhalation path is approximately 60% of vaporized dose⁽¹²⁶⁾: 10,784 mg×1.4=15,097 mcg=15 mg.

Content of active ingredient in preparation being considered: 18 mg/ml=18,000 mcg/ml.

Dose to be administered: 15,000/18,000=0.83 ml.

1 ml of preparation being considered with supplied dropper=40 drops.

Dose to be placed in electric vaporizer bowl: 40×0.83=33 drops.

Since the rate of decay of terpenes in ambient air is not known in the literature and we did not evaluate it with our device, we established arbitrarily a ground fall time of approximately 12 hours and therefore the dose was repeated after 12 hours with the use of the second electric vaporizer. Accordingly, although it was not possible to assess in absolute values the antidepressant activity of the terpenes used, however it was possible to establish a relationship between the effectiveness of the inhaled terpenes (tested individually, in association and at various known concentrations) with respect to imipramine, the same experimental conditions having been maintained during all the tests.

The second test (test no. 9 of Table 1 (FIG. 3)) was performed by doubling the dose.

Subsequent tests were performed with the individual components (35 mg repeated after 12 hours), in pairs (35 mg+35 mg, repeated after 12 hours) and then all three together (35+35+35 mg repeated after 12 hours).

The subsequent doses were prepared in order to observe whether there is synergy among the three substances and whether there is full or partial agonistic behavior, so as to observe whether a dose-effect curve of the logarithmic type occurs (as the dose doubles, the effect increases by 10%).

The treatment was performed 30 minutes before the test for the control group and for the imipramine group intraperitoneally. The third group was subject, after the T₀ test, to vaporization for 24 hours (12+12 hours) of the anonymous substance being considered.

All the expected manifestations of the protocol were reported and any adverse manifestations that might have occurred in the 72 hours that followed the test were also reported.

Clinical observations were performed during the testing period in order to check for the onset of any adverse reactions.

### Results and Discussion

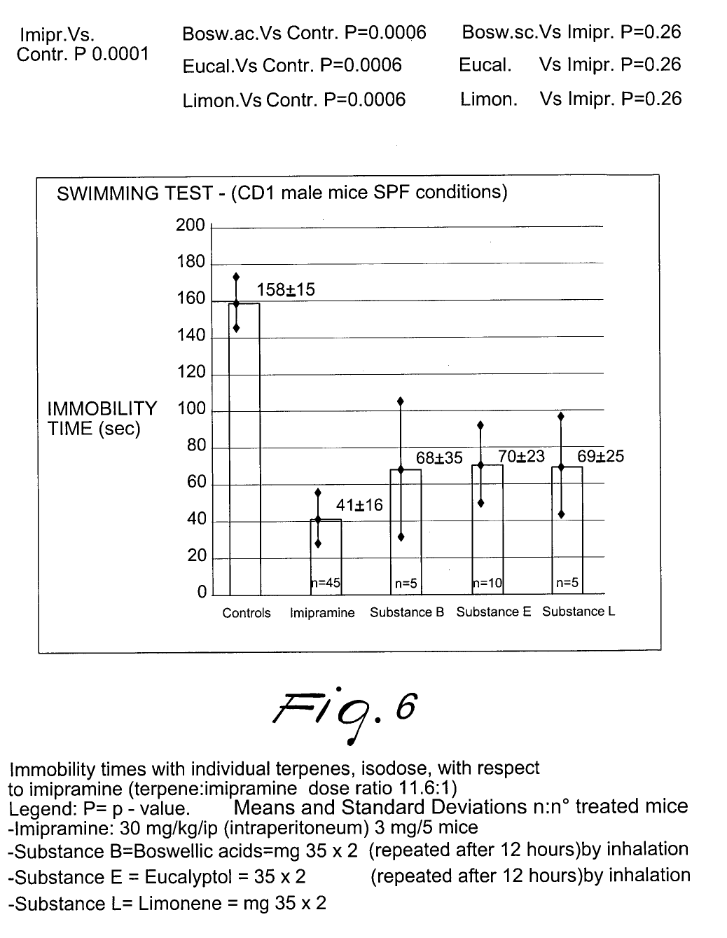

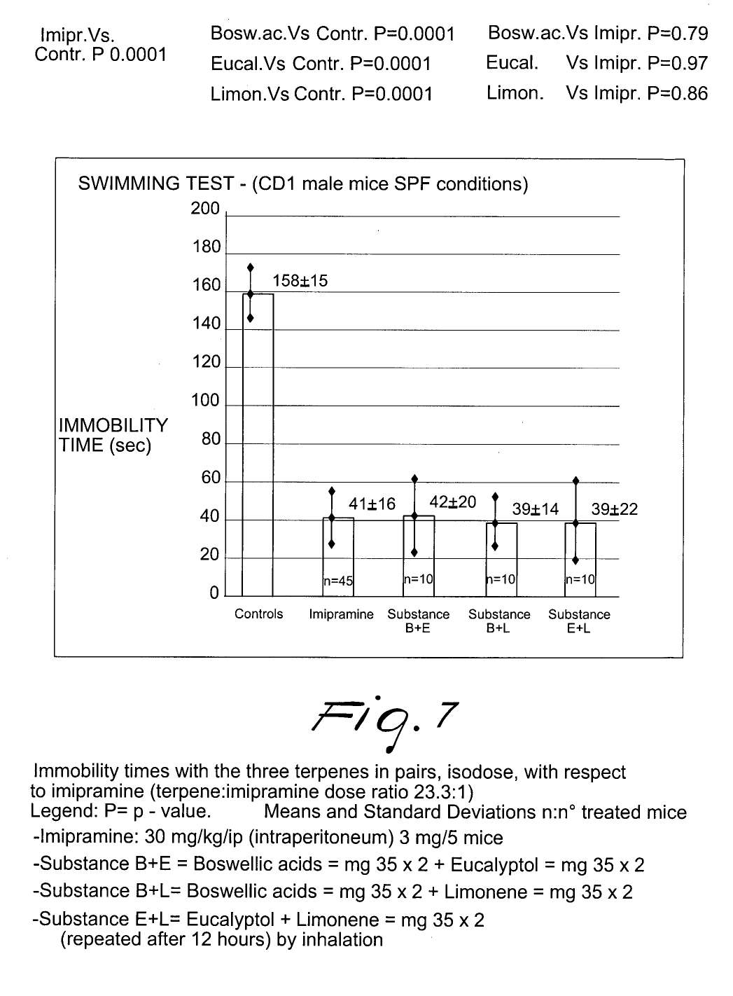

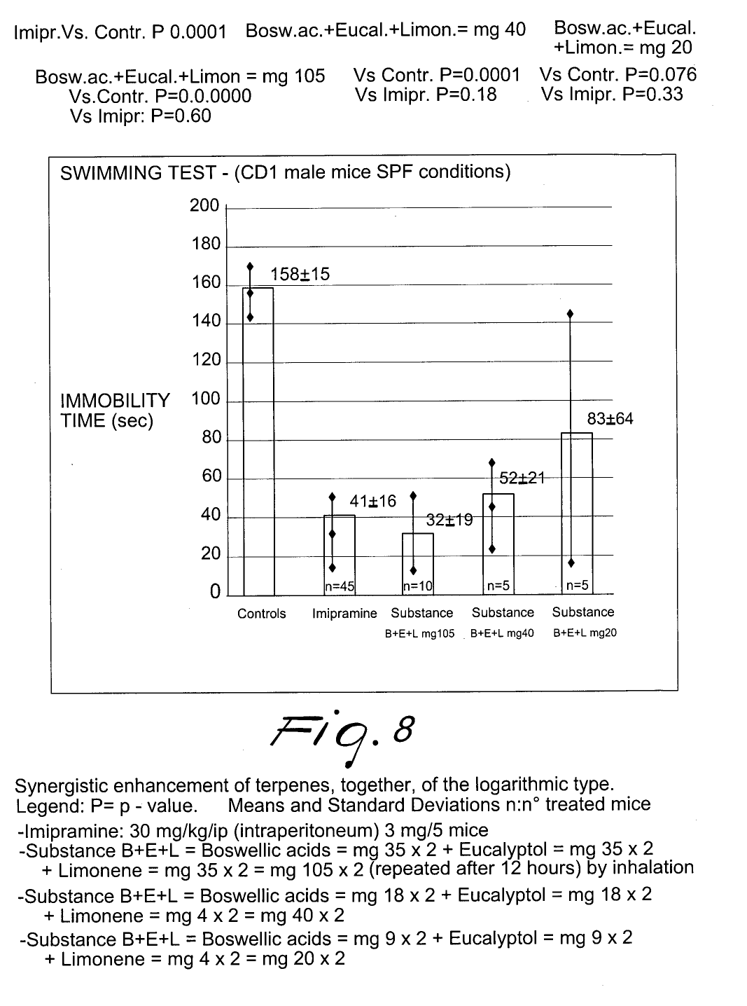

FIGS. 6, 7 and 8 illustrate the histograms of the tests conducted.

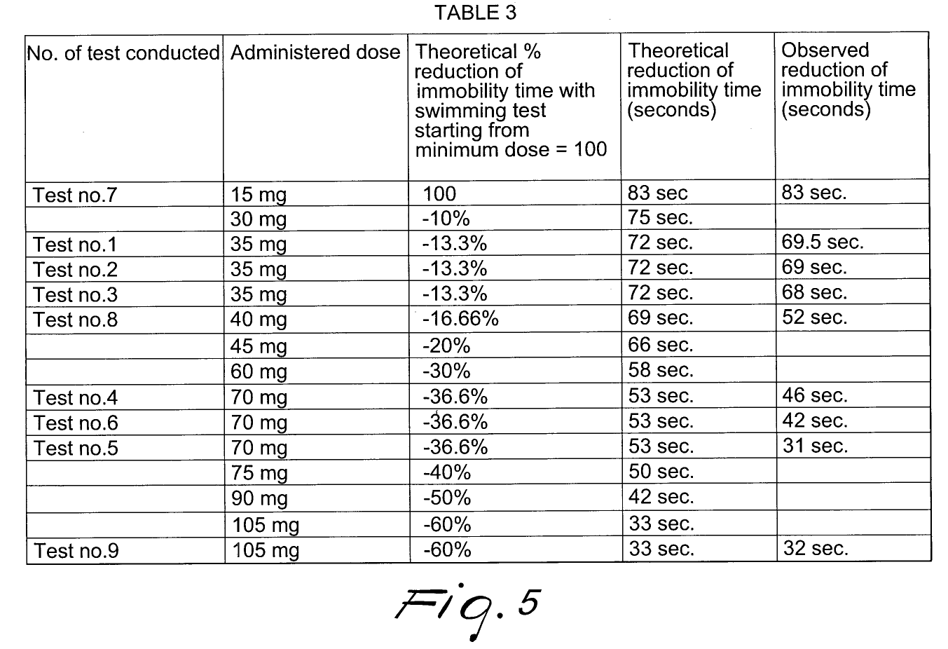

The results achieved are summarized in Table 2 (FIG. 4).

These results become more significant if the immobility times of the individual tests are proportioned to the total mean immobility time of imipramine (41.11±16.12), i.e., referred to the mean of all the tests conducted (=45 mice) and to the total mean time of the control tests.

These data allow the following statements:

1. The immobility times of the control mice group and of the group of mice treated with imipramine (9 tests and a total of 45 mice used for each group) are homogeneous with very small standard deviations.

2. The difference between the immobility times of the control group (158.06±15.20) and the imipramine group (41.11±16.12) is approximately 4:1, confirming the validity of the swimming test.

3. The boswellic acids that we tested [11-keto-beta-boswellic acid (KBA) and acetyl-11-keto-beta-boswellic acid (AKBA)], eucalyptol and limonene showed an evident antidepressant activity, apparently equal to each other as shown in Table 2—FIG. 4.

4. The isoeffect dose with respect to imipramine in the swimming test, i.e., the dose at which the individual single inhalation of KBA+AKBA, of limonene and of 1,8-cineol yielded approximately the same immobility time with respect to imipramine, was 70 mg with a ratio with respect to imipramine of approximately 23.3:1 (70:3), =700 mg/kg of body weight of terpenes versus 30 mg/kg of imipramine; more precisely, 700×0.6=420 mg/kg, taking into account the mean absorption factor of terpenes (60%) when administered by inhaling⁽¹²⁶⁾, a value slightly higher than observed in the literature for 1,8-cineol (200-400 mg/kg in the mouse)⁽¹²¹⁾.

5. The optimum dose for inhaling in terms of the antidepressant effect appears to be the one indicated above, i.e., a total of 70 mg, which corresponds to 40 mg/kg of body weight of the mice: Table 2—FIG. 4.

6. A higher dose, such as the administered one of 105 mg, which correspond to 21 mg/kg of body weight, does not increase its effectiveness significantly.

7. The dose-effect comparison with imipramine of KBA+AKBA, 1.8-cineol and limonene individually, associated with each other in pairs, as well as all three together, with a mutual ratio of 1:1:1, demonstrates a full agonist action, with an intrinsic activity equal to 1. Indeed, not only is a dose-effect curve of the logarithmic type demonstrated (as the dose doubles, a 10% effect increase occurs), but there is even a synergistic enhancement, with shorter immobility times than the theoretical values, albeit not in a significant way.

8. Synergy with enhancement is observed also with a different formulation, tested with a boswellic acid, eucalyptol, limonene ratio of 1:1:0.2 respectively. Indeed, according to personal experience, the presence of limonene can be reduced to approximately 1/5 without altering the antidepressant properties of the preparation. This is due presumably to the different mechanism of action of terpenes, which is not only added but is enhanced (see histograms of FIGS. 6, 7 and 8).

9. The same applies for the component 1,8-cineol, so that the composition of the preparation can vary from 1:1:1 to 1:0.20:0.20.

10. The 1:5 KBA/AKBA ratio of the hydroalcoholic extract, use in the tests (containing 2.9 mg/ml 11-keto-beta-boswellic acid and 14.6 mg/ml acetyl-11-keto-beta-boswellic acid; total boswellic acids 17.5 mg/ml) is also suitable to apply an antidepressant action.

11. The KBA/AKBA ratio can vary from 0.2:1 to 1:1.

12. It is likely that other boswellic triterpenes also have an antidepressant action, which is claimed.

### B. Clinical Study of Antianxiety Action of Boswellic Acids

Boswellic acids also have an antianxiety action. For this purpose, KBA and AKBA were administered to 5 healthy volunteer subjects affected by anxiety neurosis, with somatic symptoms [feeling of anxiety, fear without reason, tiredness and asthenia, tachycardia, difficulty in going to sleep, nightmares, sometimes a feeling of respiratory difficulty, sweating hands] without a history of panic attacks, never treated with psychopharmaceuticals, with a percentage of anxiety of more than 50% but with a percentage of depression within the limits of normality, i.e., less than 35%, according to the WWK Zung assessment scales administered to them⁽¹³²⁻¹³⁴⁾.

### Material and Method

The preparation according to test no. 1 (containing 17.5 mg/ml of boswellic acids) was used.

Elettromatt Turispharma electric vaporizers were used to vaporize the terpenes. To avoid dispersing most of the drug into the environment, the instrument was rested against the upper lip and inhaled with the nose, while the mouth was shut.

The dose usually administered with vaporization was 2 ml=35 mg once per day, for two weeks.

This is an assuredly harmless dose, considerably lower than that commonly used in humans both orally and by inhaling. Indeed, the dose actually taken was respectively approximately 13 mg of the vaporized 35 mg; taking into account that pulmonary uptake is approximately 70% and that almost half of the vaporized dose is not inhaled while exhaling and is dispersed into the environment. The dosage of boswellic acids commonly used orally varies from 125 to 250 mg 2-3 times per day (300-750 mg/day⁽¹³⁵⁾, i.e., 23-58 times greater).

The considerable difference in dosage between oral pathway and pulmonary pathway demonstrates that for terpenes, particularly for boswellic acids:
the inhalation pathway is the elective one, since it avoids hepatic first pass metabolism in addition to the topical action on the respiratory tract;
it does not involve transport by means of Pgp glycoproteins, which inhibit intestinal absorption and passage from the circulatory stream to the cerebrospinal liquor;
presumably, blood absorption is instead facilitated by olfactory binding proteins;
accordingly, nasal administration (such as for example by aerosol, spray, ointment, powder) is the most effective.

For informed consent, each subject was informed in detail regarding the content of the preparation, the data of the specific literature, the expected results, any side effects, always obtaining his or her consent.

Clinical tests during and after treatment were performed by the undersigned, with a report to the treating physician.

### Results

in all cases there was a rapid symptom improvement, already after a few days, with a feeling of well-being;
clinical stabilization was achieved already after one week;
symptom remission was complete and not partial and persisted throughout the period of observation for the following two months;
the reduction in the score and percentage of anxiety according to the Zung assessment scale was found to be below 40% in all subjects;
no side effects.

### C. Clinical Study of Antianxiety and Antidepressant Action of the Terpene Preparation

### Material, Method and Personal Case Series

A preparation according to test no. 8 of Table 2 (containing per ml: 9 mg boswellic acids+9 mg 1,8-cineol+2 mg limonene; total terpenes 20 mg) was used.

Elettromatt Turispharma electric vaporizers were used to vaporize the terpenes. To avoid dispersing most of the drug into the environment, the instrument was placed against the upper lip and inhaled with the nose while the mouth was shut.

The dose usually administered with vaporization was 2 ml=40 mg, once a day, generally in cycles of two weeks followed by one week of suspension, for two months (total: 6 weeks of therapy with two intervals of one week each). In some cases, the dose was prescribed twice per day (=80 mg/day) at the same rate; in particular cases, the therapy was continued in cycles for many months.

For informed consent, each patient was informed in detail regarding the content of the preparation, the expected results and any side effects. Before beginning the treatment, the treating general physician and/or specialist was informed in writing, providing explanations as to the opportuneness of the treatment, information on the content of the preparation and on the data of the specific literature, always obtaining consent therefrom.

Terpene treatment was always complementary to the treatment in progress, with the patient in a clinical steady state and under continuous and unchanged pharmacological treatment for months or years; in many cases the patients had not begun any treatment and the clinical conditions of anxiety and/or depression had only been diagnosed and were not severe, such as to allow to begin the terpene treatment without any risk.

Clinical tests during and after treatment were performed by the undersigned, with a report to the treating physician. Any reduction in the dose of psychopharmaceuticals was always prescribed by the treating physician or by the specialist.

Questionnaires to evaluate depression and anxiety were administered to the patients prior to the treatment. In particular:
Zung's depression assessment scale; the study included only those who had a score of more than 55/80, which corresponded to a percentage of depression of more than 70%;
Zung's anxiety assessment scale; the study included only those who had a score of more than 55/80, with an anxiety percentage of more than 70%;
Nijmegen's questiormaire⁽¹³⁶⁾; only subjects who had a score of 30/64 or more were included;
for the panic attack diagnosis, the questionnaire of the American Psychiatric Association was used, requiring the presence of at least four of the following characteristic symptoms: 1. respiratory oppression and hyperventilation; 2. palpitations; 3. chest pains; 4. sense of suffocation and asphyxia; 5. vertigo; 6. sense of unreality; 7. paresthesias; 8. Feelings of cold or heat; 9. sweating; 10. lipothymia; 11. tremors and convulsions; 12. fear of dying or of becoming mad.

The case series included 64 cases, distributed as follows:

Plate sclerosis: 1 case

Alzheimer's disease: 2 cases

Parkinson's disease: 2 cases

schizophrenia: 1 case

COPD: 20 cases

gastroesophageal reflux: 13 cases

OSAS: 6 cases

anxiety and depression (recurring anxiety, chronic anxiety): 12 cases

anxiety with panic attacks: 5 cases.

The small size of the sample, however, prevents reliable statistical analysis but allows some conclusions:

1. In all cases rapid symptom improvement occurred already after a few days;

2. Clinical stabilization was achieved already after one week;

3. Symptom remission was complete and not partial (patients with apathetic response or with anxious response or worried, sleepless or with somatic symptoms);

4. Terpene therapy was effective also in reducing relapses, which were absent throughout the period of observation of over one year; in the small number of cases with resumption of symptoms, repetition of terpene therapy reestablished rapidly the previous conditions.

5. In many cases, the score reduction and the percentage of anxiety and depression according to Zung's assessment scale was found to be below 40%;

6. In the 5 people with panic attacks not under pharmacological treatment, a remission occurred which persisted throughout the period of observation (over one year);

7. It was found to be beneficial on sexuality in two male subjects (aged 61 and 70) affected by OSAS.

The following observations are also very interesting.

1. Frequency of depression in subjects with gastroesophageal reflux: this leads to the hypothesis that the pathogenesis of gastroesophageal reflux involves also central nervous dysfunctions which can induce depression and that the preparation according to the disclosure interrupts these circuits. The discovery of this is claimed;

2. The constant frequency of the gastroesophageal reflux in OSAS can be explained indeed by hypothesizing that there are, in both physiopathological conditions, common dysfunctions at the level of the central nervous system. The preparation according to the disclosure is capable of inducing a neuronal functional reset and the discovery of this is claimed;

3. For the same reasons, the terpene preparation according to the disclosure is also capable of having a beneficial influence on sexual dysfunctions in OSAS, and the discovery of this is claimed;

4. Mood stabilization, which drugs available so far do not provide: the discovery of this is claimed;

5. Enhancement of attention, concentration, behavior, together with depression and anxiety: the discovery of this is claimed;

6. Stabilization of memory, together with depression and anxiety: the discovery of this is claimed;

7. It was well tolerated even by subjects with persistent severe asthma (1 case), with intolerance to aspirin (4 cases), with Churg-Strauss syndrome (1 case);

8. It did not induce weight increase;

9. It was found to have no side effects, much less habits and addition effects;

10. All this leads to the hypothesis of a mechanism of action that is different from tricyclic antidepressants and from benzodiazepine sedatives;

11. The data related to the composition of the preparation is interesting: the synergistic enhancement of the component terpenes is performed with a 1:1:1 ratio (in this order, boswellic acids: 1,8-cineol:limonene), but also with a ratio of 1:1:0.2.

12. From what has been described, one deduces that the intended aim and objects have been achieved, providing a compound that is particularly suitable for treating depression and anxiety.

### REFERENCES

1. Kline N S. The practical management of depression. JAMA. 1964; 190: 732-740.
2. Adams and Victor. Principi di Neurologia. Mc Graw-Hill, 7^{th} Italian Ed. 2005; 1394-1417.
3. Ibidem no. 1, p. 472-6.
4. May M, Sartorius N. Disturbi depressivi. CIC Ed. Intern. Roma, 2^{nd} Ed. 2005: 20-1.
5. Pope H G, Hudson J I, Jonas J M, Yurgelun-Todd D. Bulimia treated with imipramine: a placebo-controlled double-blind study. Am J Psychiatry. 1983; 140:554-8.
6. Wessely S, Powell R. Fatigue syndromes: A comparison of chronic “postviral” fatigue with neuromuscular and effective disorders. J Neurol Neurosurg Psychiatry. 1989; 52: 940-8.
7. Stahl S. Psicofarmacologia essenziale. Basi neuroscientifiche e applicazioni pratiche. Italian Ed. CEI. 2002; p. 563.
8. McAlpine's Multiple Sclerosis. Churchill Livingstone Elsevier, Fourth Edition. 2005; p. 320.
9. Harrison. Principi di Medicina Interna. McGraw-Hill 14^{th} Ed. 1998; p. 2832-4.
10. Asnaashari A M, Talaei A, Haghigh B. Evaluation of psychological status in patients with asthma and COPD. Iran J Allergy Immunol. 2012; 11: 65-71.
11. Pommer A M, Pouwer F, Denollet J and Pop V J M. Managing co-morbid depression and anxiety in primary care patients with asthma and/or chronic obstructive pulmonary disease: study protocol for a randomized controlled trial. Trials. 2012; 13: 6-12.
12. Wheeler E O, White P D, Reed E W, Cohen M E. Neurocircolatory asthenia (anxiety neurosis, effort syndrome, neurasthenia). JAMA. 1950; 142:878-889.
13. Bourin M. Augmentation effect of combination therapy of aripiprazole and antidepressants on forced swimming test in mice. Psychopharmacology. 2009; 206: 97-107.
14. Carroll B J, Feinberg M, Greden J F, Tarika J, Albala A A, Haskett R F, James N M, Kronfol Z, Lohr N, Steiner M, de Vigne J P, Young E. A. Arch Gen Psychiatry. 1981; 38:15-22.
15. Kramer M S, Cutler N, Feighner J, et al. Distinct mechanism for antidepressant activity by blockade of central substance P receptors. Science. 1998; 281: 1640-5.
16. Bourin M. The role of sodium channels in the mechanism of action of antidepressants and mood stabilizers. Curr Drug Targets. 2009; 10: 1052-60.
17. Schlesser M A, Winokur G, Sherman B M. Hypothalamic-pituitary-adrenal axis activity in depressive illness. Arch Gen Psychiatry. 1980; 37: 737-743.
18. Chen B, Dowlatshahi D, MacQueen G M, Wang J F, Young L T. Increased hippocampal BDNF immunoreactivity in subjects trated with antidepressant medication. Biol Psych. 2001; 50:260-5.
19. Gold S M, Irwin M R. Depression and immunity: inflammation and depressive symptoms in multiple sclerosis. Neurol Clin. 2006; 24: 507-511.
20. Lesch K P, Bengel D, Heils A, Sabol S Z, Greenberg B D, Petri S, Benjamin J, Müller C R, Hamer D H, Murphy D L. Association of anxiety-related traits with a polymorphism in the serotonin transporter gene regulatory region. Science. 1996; 274: 1527-31.
21. Ibidem no. 7, p. 305-310.
22. Ibidem no. 2, p. 522.
23. Carr G V, Schechter L E. Antidepressant and anxiolytic effects of selective 5-HT6 receptor agonists in rats. Psychopharmacology 2011; 213: 499-507.
24. Moussaieff A, Rimmerman N, Bregman T, Straiker A, Felder C C, Shoham S, Kashman Y, Huang S M, Lee H, Shohami E, Mackie K, Caterina M J, Walker J M, Fride E, and Mechoulam R. Incensole acetate, an incense component, elicits psychoactivity by activating TRP3 channels in the brain. FASEB J. 2008; 22: 3024-34.
25. Sharma R, Singh S, Singh G D, Khajuria A, Sidiq T, Singh S K, Chashoo G, Pagoch S S, Kaul A, Saxena A K, Johri R K, Taneja Sc. In vivo genotoxicity evaluation of a plant based antiarthritic and anticancer therapeutic agent Boswellic acids in rodents. Phytomedicine. 2009; 16(12): 1112-8.
26. Lalithakumari K, Krishnaraju A V, Sengupta K, Subbaraju G V, Chatterjee A. Safety and toxicological evaluation of a novel, standardized 3-0-acetyl-11-keto-beta-boswellic acid (AKBA)-enriched Boswellia serrata extract (5-Loxin®). Toxicol Mech Methods. 2006; 16(4): 199-226.
27. Wang C, Xia L, Song Z, Li Q, Wang C, Zeng L, Liu Z. Determination of five boswellic acids in Boswellia serrata. Zhongguo Zhong Yao Za Zhi. 2011; 36(10): 1330-3.
28. Sharma S, Thawani V, Hingorani L, Shrivastava M, Bhate V R, Khiyani R. Pharmacokinetic study of 11-keto-beta boswellic acid. Phytomedicine. 2004; 11(2-3): 255-60.
29. Kruger P, Daneshfar R, Eckert G P, Klein J, Volmer D A, Bahr U, Müller W E, Karas M, Schubert-Zsilavecz M, and Abdel-Tawab M. Metabolism of boswellic acids in vitro and in vivo. Drug Metab Dispos. 2008; 36(6): 1135-42.
30. Weber C C, Reising K, Müller W E, Schubert-Zsilavecz M, and Abdel-Tawab M. Modulation of Pgp function by boswellic acids. Planta Med. 2006; 72(6): 507-13.
31. Kruger P, Kanzer J, Hummel J, Fricker G, Schubert-Zsilavecz M, Abdel-Tawab M. Permeation of Boswellia extract in the Caco-2 model and possible interactions of its constituents KBA and AKBA with OATP1B3 and MRP2. Eur J Pharm Sci. 2009; 36 (2-3): 275-84.
32. Sharma A, Gupta N K, Dixit V K. Complexation with phosphatidyl choline as a strategy for absorption enhancement of boswellic acid. Drug Deliv. 2010; 17(8): 587-95.
33. Sterk V, Büchele B, Simmet T. Effect of food intake on the bioavailability of boswellic acids from a herbal preparation in healthy volunteers. Planta Med. 2004; 70(12): 1155-60.
34. Johnston M, Zakharov A, Armstrong D. Subarachnoid injection of Microfil reveals connections between cerebrospinal fluid and nasal lymphatics in the non-human primate Neuropathol Appl Neurobiol. 2005; 31(6): 632-40.
35. Ammon H P. Modulation of the immune system by Boswellia serrata extracts and boswellic acids. Phytomedicine. 2010; 17(11): 862-7.
36. Kumar A, Shah B A, Singh S, Hamid A, Singh S K, Sethi V K, Saxena A K, Singh J, Taneja S C. Acyl derivatives of boswellic acids as inhibitors of NF-kB and STATs. Bioorg Med Chem Lett. 2012; 22(1): 431-5.
37. Cao H, Yu R, Choi Y, Ma Z Z, Zhang H, Xiang W, Lee D Y W, Berman B M, Moudgil K D, Fong H H S, and van Breemen R B. Discovery of cyclooxygenase inhibitors from medicinal plants used to treat inflammation. Phamacol Res. 2010; 61(6): 519-24.
38. Siemoneit U, Koeberle A, Rossi A, Dehm F, Verhoff M, Reckel S, Majer T J, Jauch J, Northoff H, Bernhard F, Doetsch V, Sautebin L and Werz O. Inhibition of microsomal prostaglandin E₂ synthase-1 as a molecular basis for the anti-inflammatory actions of boswellic acids from frankincense. British Journal of Pharmacology. 2011; 162: 147-162.
39. Tausch L, Henkel A, Siemoneit U, Poeckel D, Kather N, Franke L, Hofmann B, Schneider G, Angioni C, Geisslinger G, Skarke C, Holtmeier W, Beckhaus T, Karas M, Jauch J, Werz O. Identification of human cathepsin as a functional target of boswellic acids from the anti-inflammatory remedy frankincense. J Immunol. 2009; 183(5): 3433-42.
40. Roy S, Khanna S, Krishanaraju A V, Subbaraju G V, Yasmin T, Bagchi D, Sen C K. Regulation of vascular responses to inflammation: inducible matrix metalloproinase-3 expression in human microvascular endothelial cells is sensitive to antiinflammatory Boswellia. Antioxid Redox Signal. 2006; 8(3-4): 653-60.
41. Henkel A, Kather N, Winch B, Northoff H, Jauch J, Werz O. Boswellic acids from frankincense inhibit lipopolysaccharide functionality through direct molecular interference. Biochem Pharmacol. 2012 83(1): 115-21.
42. Saraswati S, Pandey M, Mathur R, Agrawal S S. Boswellic acid inhibits inflammatory angiogenesis in a murine sponge model. Microvasc Res. 2011 82(3): 263-8.
43. Ammon H P. Modulation of the immune system by Boswellia serrata extracts and boswellic acids. Phytomedicine. 2010; 17(11): 862-7.
44. Wang H, Syrovets T, Kess D, Blichele B, Hainzl H, Lunov O, Weiss J M, Scharffetter-Kochanek K, and Simmet T. Targeting N F-kB with a natural triterpenoid alleviates skin inflammation in a mouse model of psoriasis. J Immunol. 2009; 183(7): 4755-63.
45. Ali E N and M ansour S Z. Boswellic acids extract attenuates pulmonary fibrosis induced by beleomycin and oxidative stress from gamma irradiation in rats. Chinese Medicine. 2011; 6: 36-50.
46. Wildfeuer A, Neu I S, Safayhi H, Metzger G, Wehrmann M, Vogel U, Ammon H P. Effects of boswellic acids extracted from a herbal medicine on the biosynthesis of leukotrienes and the course of experimental autoimmune encephalomyelitis. Arzneimittelforschung. 1998; 48(6): 668-74.
47. Raja A F, Ali F, Khan I A, Shawl A S and Arora D S. Acetyl-11-keto-β-boswellic acid (AKBA); targeting oral cavity pathogens. BMC Research Notes. 2011, 4: 406.
48. Raja A F, Ali F, Khan I A, Shawl A, Arora D S, Shah B A, Taneja S C. Antistaphylococcal and biofilm inhibitory activities of acetyl-11-keto-f3-boswellic acid from Boswellia serrata. BMC Microbiology. 2011; 11: 54-62.
49. Safe S H, Prather P L, Brents L K, Chadalapaka G, Jutooru I. Unifying mechanism of action of the anticancer activities of triterpenoids and synthetic analogs. Anticancer Agents Med Chem. 2012; May 2.
50. Yadav V R, Prasad S, Sung B, Kanappan R, and Ahggarwal B B. Targeting inflammatory pathways by triterpenoids for prevention and treatment of cancer. Toxins (Basel). 2010; 2(10): 2428-66.
51. Park B, Prasad S, Yadav V, Sung B, Aggarwal B B. Boswellic acid suppresses growth and metastasis of human pancreatic tumors in an orthotopic nude mouse model through modulation of multiple targets. PLOS one. 2011; 6(10): e26943.
52. Chashoo G, Singh S K, Sharma P R, Mondhe D M, Hamid A, Saxena A, Andotra S S, Shah B A, Qazi N A, Taneja S C, Saxena A K. A propylloxy derivative of 11-keto-β-boswellic acid induces apoptosis in HL-60 cells mediated through topisomerase I & II inhibition. Chem Biol Interact. 2011; 189(1-2): 60-71.
53. Sankpal U T, Goodison S, Abdelrahim M, Basha R. Targeting Sp1 transcription factors in prostate cancer therapy. Med Chem. 2011; 7(5): 518-25.
54. Bhushan S, Malik F, Kumar A, Isher H K, Kaur I P, Taneja S C, Singh J. Activation of p53/p21/PUMA alliance and disruption of PI-3/Akt in multimodal targeting of apoptotic signaling cascades in cervical cancer cells by a pentacyclic triterpenediol from Boswellia serrata. Mol Carcinog. 2009; 48(12): 1093-108.
55. Shen Y, Takahashi M, Byun H M, Link A, Shama N, Balaguer F, Leung H C, Boland R, Goel A. Boswellic acid induces epigenetic alterations by modulating DNA methylation in colorectal cancer cells. Cancer Biol Ther. 2012; 13(7): 542-52.
56. Agrawal S S, Saraswati S, Mathur R, Pandey M. Antitumor properties of boswellic acid against Ehrlich ascites cells bearing mouse. Food Chem Toxicol. 2011; 49(9): 1924-34.
57. Winking M, Sarikaya S, Rahmanian A, Jödicke A, Boker D K. Boswellic acids inhibit glioma growth: a new treatment option? J Neurooncol. 2000; 46(2): 97-103.
58. Kirste S, Treier M, Wehrle S J, Becker G, Abdel-Tawab M, Gerbeth K, Hug M J, Lubrich B, Grosu A L, Momm F Boswellia serrata acts on cerebral edema in patients irradiated for brain tumors: a prospective, randomized, placebo-controlled, double-blind pilot trial. Cancer. 2011; 117(16): 3788-95.
58. Park Y S, Lee J H, Bondar J, Harwalkar J A, Safayhi H, Golubic M. Cytotoxic action of acetyl-11-keto-beta-boswellic acid (AKBA) on meningioma cells. Planta Med. 2002; 68(5): 397-401.
59. Karima O, Riazi G, Yousefi R, Moyahedi A A. The enhancement effect of beta-boswellic acid on hippocampal neurites outgrowth and branching (an in vitro study). Neurol Sci. 2010; 31(3): 315-20.
60. Bishnoi M, Patil C S, Kumar A, Kulkarni S K. Co-administration of acetyl-11-keto-beta-boswellic acid, a specific 5-lipoxygenase inhibitor, potentiates the protective effect of COX-2 inhibitors in kainic acid-induced neurotoxicity in mice. Pharmacology. 2007; 79(1): 34-41.
62. Bishnoi M, Patil C S, Kumar A, Kultarni S K. Potentiation of antinociceptive effect of NSAIDs by specific lipoxygenase inhibitor, acetyl 11-keto-beta boswellic acid. Indian J Exp Biol. 2006; 44(2): 128-32.
63. Maia J L, Lima-Júnior R C, Melo C M, David J P, David J M, Campos A R, Santos F A, Rao V S. Oleanolic acid, a pentacyclic triterpene attenuates capsaicin-induced nociception in mice: possible mechanisms. Pharmacol Res. 2006; 54(4): 282-6.
64. Lima-Júnior R C, Oliveira F A, Gungel L A, Cavalcante I J, Santos K A, Campos D A, Vale C A, Silva R M, Chaves M H, Rao V S, Santos F A. Attenuation of visceral nociception by alpha- and beta-amyrin, a triterpenoid mixture isolated from the resin of Proium heptaphyllum, in mice. Planta Med. 2006; 72(1): 34-9.
65. Chen Y, Han T, Rui Y, Yin M, Qin L, Zheng H. Effects of total triterpenes of Centella asiatica on the corticosterone levels in serum and contents of monoamine in depression rat brain. Zhong Yao Cai. 2005; 28(6): 492-6.
66. Chen H, Chan K K, Budd T. Pharmacocinetics of d-limonene in the rats by G C-M S assay. J Pharm Biomed Anal. 1998; 17(4-5): 631-40.
67. Falk-Filipsson A, Löf A, Hagberg M, Hjelm E W, Wang z. D-limonene exposure to humans by inhalation: uptake, distribution, elimination, and effects on the pulmonary function. J Toxicol Environ Health. 1993; 38(1): 77-88.
68. Larsen St, Hougaard K S, Hammer M, Alarie Y, Wolkoff P, Clausen P A, Wilkins C K, Nielsen G D. Effects of R-(+)- and S-(−)-limonene on the respiratory tract in mice. Hum Exp Toxicol. 2000; 19(8): 457-66.
69. Abdullah G Z et al. In vitro permeation and in vivo anti-inflammatory and analgesic properties of nanoscaled emulsions containing ibuprofen for topical delivery. International Journal of Nanomedicine. 2011:6 387-396.
70. Miller J A et al. Determination of d-limonene in adipose tissue by gas chromatography-mass spectrometry. J Chromatogr Analyt Technol Biomed Life Sci. 2008; 870 (1): 68-73.
71. Hamada M, Uezu K, Matsushita J. Distribution and immune responses resulting from oral administration of d-limonene in rats. J Nutr Sci Vitaminol (Tokio). 2002; 48(2): 155-160.
72. Crowel P L, Lin S, Vedejs E, Gould M N. Identification of metabolites of the antitumor agent d-limonene capable of inhibiting protein isoprenylation and cell growth. Cancer Chemother Pharmacol. 1992; 31: 205-212.
73. Kim J H, Lee H J, Jeong S J, Lee M H, Kim S H. Essential oil of Pinus koraiensis leaves exerts antihyperlipidemic effects via up-regulation of low-density lipoprotein receptor and inhibition of acyl-coenzyme A: cholesterol acyltransferase. Phytother Res. 2012; doi: 10.1002/ptr.3734.
74. Sun J. D-limonene: safety and clinical applications. Alternative Medicine Review. 2007; 12 (3): 259-264.
75. Yoon W J, Lee N H and Hyun C-G. Limonene suppresses lipopolysaccharide-induced production of nitric oxide, prostaglandin E₂, and pro-inflammatory cytokines in RAW 264.7 macrophages. J Oleo Sci. 2010; 59 415-421.
76. Chaudhary S C, Siddiqui M, Athar M, Alam M S. D-Limonene modulates inflammation, oxidative stress and Ras-ERK pathway to inhibit murine skin tumorigenesis. Hum Exp Toxicol. 2012; 31(8): 798-811.
77. Fernandez-Bedmar Z, Anter J, de La Cruz-Ares S, Mufioz-Serrano A, Alonso-Moraga A, Pérez-Guisado J. Role of citrus juices and distinctive components in the modulation of degenerative processes: genotoxicity, antigenotoxicicity, cytotoxicity, and longevity in Drosophila. J Toxicol Environ Health. 2011; 74(15-16): 1052-66.
78. Moraes T M, Kushima H, Moleiro F C, Santos Rc, Rocha L R, Marques M O, Vilegas W, Hiruma-Lima C A. Effects of limonene and essential oil from Citrus aurantium on gastric mucosa: role of prostaglandins and gastric mucus secretion. Chem Biol Interact. 2009; 180(3): 499-505.
79. Heuberger E, Hongratanaworakit T, Böhm C, Weber R, Buchbauer G. Effects of chiral fragrances on human autonomic nervous system parameters and self evaluation. Chem Senses. 2001, 26(3): 281-92.
80. Cha J H, LeeSH, Yoo Y S. Effects of aromatherapy on changes in the autonomic nervous system, aortic pulse wave velocity and aortic augmentation index in patients with essential hypertension. J Korean Acad Nurs. 2010; 40(5): 705-713.
81. Lu M, Battinelli L, Daniele C, Melchioni C, Salvatore G, Mazzanti G. Muscle relaxing activity of Hysoopus officinalis essential oil on isolated intestinal preparations. Planta Med. 2002; 68(3): 213-6.
82. De Sousa D P et al. Structure and spasmolytic activity relationships of monoterpene analogues found in many aromatic plants. Z Naturforsch C. 2008; 63(11-12): 808-12.
83. Menichini F, Tundis R, Liozzo M R, Bonesi M, Marrelli M, Statti G A, Menichini F, Conforti F. Acetylcholinesterase and butyrylcholinesterase inhibition of ethanolic extract and monoterpenes from Pimpinella anisoides V Brig. (Apiaceae). Fitoterapia. 2009; 80(5): 297-300.
84. Zhou W et al. Components of lemon essential oil attenuate dementia induced by scopolamine. Nutr Neurosci. 2009; 12(2): 57-64.
85. Tundis R, Liozzo M R, Bonesi M, Menichini F, Mastellone V, Colica C, Menichini F. Comparative study on the antioxidant capacity and cholinesterase inhibitory activity of Citrus aurantifolia Swingle, C. aurantium L., and C. bergamia Risso and Poit. Peel essential oils. J Food Sci. 2012; 77(1): H40-6.
86. Golshani S, Karamkhani F, Monsef-Esfehani H R, Abdollahi M. Antinociceptive effects of the essential oil of Dracocephalum kotschyi in the mouse writhing test. Jharm Sci 2004; 7(1): 76-79.
87. Faturi C B et al. Anxiolytic-like effect of sweet orange aroma in Wistar rats. Prog Neuropsycopharmacol Biol Psichiatry. 2010; 34(4): 605-9.
88. Zhou W, Miyuki Y and Yokogoshi H. Sub-chronic effects of s-limonene on brain neurotransmitter levels and behavior of rats. J Nutr Sci Vitaminol. 2009; 55, 367-373.
89. Komiya M. Lemon oil vapor causes an anti-stress effect via modulating the 5-H T and D A activities in mice. Behav Brain Res. 2006; 172(2): 240-9.
90. Komori T, Fujiwara R, Tanida M, Nomura J. Potential antidepressant effects of lemon odor in rats. Eur Psychopharmacol. 1995; 5: 477-80.
91. Komori T, Matsumoto T, Motomura E and Shiroyama T. The sleep-enhancing effect of valerian inhalation and sleep-shortening effect of lemon inhalation. Chem. Senses. 2006; 31: 731-7.
92. Neto A C et al. The role of polar phytocomplexes on anticonvulsant effects of leaf of Lippia alba (Mill.) N.E. Brown chemotypes. J Pharm Pharmacol. 2009; 61(7): 933-9.
93. Jager W, Nas\check{e}l B, Nas\check{e}l C, Binder R, Stimpfl T, Vycudilik W and Buchbauer G. Pharmacokinetics studies of the fragrance compound 1,8-cineol in humans during inhalation. Chem. Senses. 1996; 21: 477-480.
94. Kovar K A, Gropper B, Friess D & Ammon H P T. Blood levels of 1,8-cineol and locomotor activity of mice after inhalation and oral administration of rosemary oil. Planta Medica. 1987; 53: 315-8.
95. Tisserand R & Balacs T. Essential oil safety. Churchill Livingtone, U K. 1995; 2-16.
96. Jager W, Nas\check{e}l B, Nas\check{e}l C, Binder R, Stimpfl T, Vycudilik W and Buchbauer G. Pharmakinetic studies of the fragrance compound 1,8-cineol in humans during inhalation. Chemical Senses. 1996; 21: 477-480.
97. Juergens U R, Stöber M & Vetter H. Inhibition of cytokine production and arachidonic acid metabolism by eucalyptol (1.8-cineole) in human blood monocytes in vitro. Eur J Med Res. 1998; 3:508-510.
98. Juergens U R, Engelen T, Racké K, Stöber M, Gillissen A, Vetter H Inhibitory activity of 1,8-cineol (eucalyptol) on cytokine production in cultured human lymphocytes and monocytes. Pulm Pharmacol Ther. 2004; 17(5): 281-7.
99. Zhou J-y, Wang X-f, Tang F-d, Zhou J-y, Lu G-h, Wang Y, Bian R-1 Inhibitory effect of 1,8 cineol (eucalyptol) on Egr-1 expression in lipolysaccharide THP-1 cells. Acta Pharmacol Sin. 2007; 28(6): 908-12.
100. Loizzo M R, Menichini F, Tundis R, Bonesi M, Conforti F, Nadjafi F, Statti G A, Frega N G and Menichini F. In vitro biological activity of Salvia Leriifolia Benth essential oil relevant to the treatment of Alzheimer's Disease. I Oleo Sci. 2009; 58(8): 443-6.
101. Santos F A, Silva R M, Campos A R, De Arairjo R P, Lima Ernior R C, Rao V S. 1,8-cineole (eucalyptol), a monoterpene oxide attenuates the colonic damage in rats on acute TNBS-colitis. Food Chem Toxicol. 2004; 42(4): 579-84.
102. Bastos V P, Gomes A S, Lima F J, Brito T S, Soares P M, Pinho J P, Silva C S, Santos A A, Souza M H, Magalhães P J. Inhaled 1,8-cineole reduces inflammatory parameters in airways of ovalbumin-challenged Guinea pigs. Basi Clin Pharmacol Toxicol. 2011; 108(1): 34-9.
103. Juergens U R, Stöber M, Schmidt-Schilling L, Kleuver T & Vetter H. Antiinflammatory effects of eucalyptol (1,8-cineole) in bronchial asthma: inhibition of arachidonic acid metabolism in human blood monocytes ex vivo. Eur J Med Res. 1998; 3: 407-412.
104. Nascimento N R, Refosco R M, Vasconcelos E C, Kemtopf M R, Santos C F, Batista F J, De Sousa C M, Fonteles M C. 1,8-cineole induces relaxation in rat and guinea-pig airway smooth muscle. J Pharm Pharmacol. 2009; 61(3): 361-3.
105. Laude E A, Morice A H & Grattan T J. The antitussive effects of menthol, camphor and cineole in conscious guinea-pigs. Pulmonary Pharmacology. 1994; 7: 170-184.
106. Santos F A, Rao V S. 1,8-Cineol, a food flavoring agent, prevents ethanol-induced gastric injury in rats. Dig Dis Sci. 2001; 46: 331-7.
107. Kovar K A, Gropper B, Friess D & Ammon H P T. Blood levels of 1,8-cineol and locomotor activity of mice after inhalation and oral administration of rosemary oil. Planta Medica. 1987; 53: 315-8.
108. Stimpfl T, Nas\check{e}l B, Nas\check{e}l C, Binder R, Vycudilik W, Buchbauer G. Concentration of 1,8-cineole in blood during prolonged inhalation. Chemical Senses. 1995; 20: 349-350.
109. Lahlou S, Figueiredo A F, Magalhāes P J, Leal-Cardoso J H. Cardiovascular effects of 1,8-cineole, a terpenoid oxide present in many plant essential oils, in normotensive rats. Can J Physiol Pharmacol. 2002; 80(12): 1125-1131.
110. Soares M C M S, Damiani C E N, Moreira C M, Stefanon I and Vassallo D V. Eucalyptol, an essential oil, reduces contractile activity in rat cardiac muscle. Braz J Med Biol Res. 2005; 38(3): 453-461.
111. Loizzo M R, Menichini F, Tundis R, Bonesi M, Conforti F, Nadjafi F, Statti G A, Frega N G and Menichini F. In vitro biological activity of Salvia Leriifolia Benth essential oil relevant to the treatment of Alzheimer's Disease. I Oleo Sci. 2009; 58(8): 443-6.
112. Kaufmann D, Dogra A K, Wink M. Myrtenal inhibits acetylcholinesterase, a known Alzheimer target. J Pharm Pharmacol. 2011; 63(10): 1368-71.
113. Aazza S, Lyoussi B, Miguel M G. Antioxidant and antiacetylcholinesterase activities of some commercial essential oils and their major compounds. Molecules. 2011; 16(9): 7672-90.
114. Weyers W & Brodbeck R. Skin absorption of volatile oils. Pharmacokinetics Pharmazie in unserer Zeit. 1989; 18: 82-6.
115. Lim P F, Liu X Y, Kang L, Ho P C, Chan Y W, Chan S Y. Limonene GP1/PG organogel as vehicle in transdermal delivery of haloperidol. Int J Pharm. 2006; 311(1-2): 154-64.
116. Ou M C, Hsu T E, Lai A C, Lin Y T, Lin Cc. Pain relief assessment by aromatic essential oil massage on outpatients with primary dysmenorrhea: a randomized, double-blind clinical trial. J Obstet Gynecol Res. 2012; 38(5): 817-22.
117. Zalachoras I, Kagiava A, Vokou D, Theophilidis G. Assessing the local anesthetic effect of five essential oil constituents. Planta Med. 2010; 76(15): 1647-53.
118. Liapi C, Anifandis G, Chinou I, Kourounakis A P, Theodosopoulos S, Galanopoulou P. Antinociceptive properties of 1,8-Cineole and beta-pinene, from the essential oil of Eucalyptus camaldulensis leaves, in rodents. Planta Med. 2007; 73(12): 1247-54.
119. Santos F A, Rao V S. Antiinflammatory and antinociceptive effects of 1,8-cineole a terpenoid oxide present in many plant essential oils. Phytother Res. 2000; 14(4): 240-4.
120. Sherkheli M A, Benecke H, Doerner J F, Kletke O, Vogt-Eisele A K, Gisselmann G and Hatt H. Monoterpenoids induce agonist-specific desensitization of transient Receptor Potential Vanilloid-3 ion channels. J Pharm Pharmacol Sci. 2009; 12(1): 116-126.
121. Gomes P B, Feitosa M L, Silva M I, Noronha E C, Moura B A, Venancio E T, Rios E R, de Sousa D P, de Vasconcelos S M, Fonteles M M, de Sousa F C. Anxiolytic-like effect of the monoterpene 1,4-cineole in mice. Pharmacol Biochem Behay. 2010; 96(3): 287-93.
122. Satou T, Murakami S, Matsuura M, Hayashi S, Koike K. Anxiolytic effect and tissue distribution of inhaled Alpinia zerumbet essential oil in mice. Nat Prod Commun 2010; 5(1): 143-6.
123. Umezu T, Nagano K, Ito H, Kosakai K, Sakaniwa M, Morita M. Anticonflict effects of lavender oil and identification of its active constituents. Pharmacol Biochem Behay. 2006; 85(4): 713-21.
124. Machado D G, Bettio L E, Cunha M P, Capra J C, Dalmarco J B, Pizzolatti M G, Rodrigues A L. Antidepressant-like effect of the extract of Rosmarinus officinalis in mice: involvement of the monoaminergic system. Prog Neuropsychopharmacol Biol Psychiatry 2009; 33(4): 642-50.
125. Aoshima H and Hamamoto K. Potentiation of GABA_{A} receptors expressed in Xenopus Oocytes by perfume and phytoncid. Biosci. Biotechnol. Biochem 1999; 63(4): 743-48.
126. The German Working Group on Indoor Air Guidelines of the Federal Environment Agency and the States' Health Authorities. Indoor air guide values for monocyclic monoterpenes (limonene). Bundesgesundheitsblatt Gesundheitsforschung Gesundheitsschutz 2010; 53(11): 1206-15.
127. Zung W W K. A rating instrument for anxiety disorders. Psychosomatics. 1971; 12(6):371-9.
128. Zung W W K. The measurement of effects: depression and anxiety. Mod Probl Pharmacopsychiatry. 1974; 7(0): 170-88.
129. Zung W W K. A self-rating depression scale. Arch Gen Psychiatry. 1965; 12: 63-70.
130. Siddiqui M Z. Boswellia serrata, a potential antiinflammatory agent: an overview. Indian J Pharm Sci. 2011; 73(3): 255-261.
131. Chaitow L, Bradley D, Gilbert G. Multidisciplinary approaches to breathing pattern disorders. London: Churchill Livingstone, 2002:176.

1-18. (canceled)

19. A compound particularly for treating depression and anxiety, comprising the combination of at least 11-keto-beta-boswellic acid (KBA) and acetyl-11-keto-beta-boswellic acid (AKBA).

20. The compound particularly for treating depression and anxiety, according to claim 19, wherein the proportion between the KBA and AKBA boswellic acids is comprised between 1:1 and 1:5.

21. The compound particularly for treating depression and anxiety, according to claim 19, further comprising the combination of KBA with at least one other triterpene boswellic acid in a ratio comprised between 1±0.8:1±0.8.

22. The compound particularly for treating depression and anxiety, according to claim 19, further comprising the combination of AKBA with at least one other triterpene boswellic acid in a ratio comprised between 1±0.8:1±0.8.

23. The compound particularly for treating depression and anxiety, according to claim 19, further comprising the combination of at least KBA and AKBA boswellic acids with D-limonene in proportion, in the order 1 unit of KBA, (1±0.8) units of AKBA and (1±0.8) units of D-limonene.

24. The compound particularly for treating depression and anxiety, according to claim 19, further comprising the combination of at least KBA and AKBA boswellic acids with S-limonene in proportion, in the order 1 unit of KBA, (1±0.8) units of AKBA and (1±0.8) units of S-limonene.

25. The compound particularly for treating depression and anxiety according to claim 19, further comprising the combination of at least KBA and AKBA boswellic acids with S-limonene in proportion, in the order 1 unit of KBA+AKBA and (1±0.8) units of 1,8-cineol.

26. The compound particularly for treating depression and anxiety according to claim 19, further comprising the combination of at least KBA and AKBA boswellic acids with D-limonene, S-limonene and 1,8-cineol, in proportion, in the order (1±0.8) units of KBA, (1±0.8) units of AKBA and (1±0.8) units of D-limonene+S-limonene+1,8-cineol.

27. Administration of a compound according to claim 19, nasally or by any means (e.g., aerosol, spray, ointment, drops).

28. Use of a compound according to claim 19 for the manufacture of a medical-sanitary product with antidepressant action as complementary therapy and for prophylaxis of recurrences and relapses of major depression and in all particular clinical manifestations of depression, as in anorexia nervosa and bulimia, in chronic fatigue syndrome, in sexual depression in men and women, in other neurologic and psychiatric disorders, in invalidating chronic internistic disorders, in obstructive sleep apnea syndrome (OSAS), in iatrogenic depression, in gastroesophageal reflux disease (GERD), in mixed anxiety-depressive syndrome.

29. Use of a compound according to claim 19 for the manufacture of a medical-sanitary product for prophylaxis of relapses and recurrences of generalized anxiety disorder (GAD) and in all particular clinical manifestations such as in recurring subclinical anxiety, chronic subclinical anxiety, persistent anxiety and anxious depression, in panic attack disorders, in all varieties of neurosis, in stress and in stress-related syndromes.

30. Use of a compound according to claim 19 for the manufacture of a medical-sanitary product for therapeutic prophylaxis for mood stabilization, for enhancement of attention, concentration, behavior and for memory stabilization, in addition to the anti-anxiety and antidepressant action, useful especially in psychiatric diseases such as schizophrenia.

31. Use of a compound according to claim 19 for the manufacture of a medical-sanitary product for therapeutic prophylaxis in sexual disorders such as libido reduction and male impotence, postpartum depression, female perimenopausal depression, male and female infertility both due to depression and due to dysfunctions of the hypothalamus for their rebalancing action on neurotransmission.

32. Use of a compound according to claim 19 for the manufacture of a medical-sanitary product for therapeutic prophylaxis for neurotrophic, neuroprotective action improving cerebral perfusion and together with the anti-anxiety and antidepressive action in degenerative diseases (such as Alzheimer's disease, Huntington's chorea, Parkinson's disease, amyotrophic lateral sclerosis) and in demyelinating diseases of the nervous system (such as multiple sclerosis) together with anxiety and depression.

33. Use of a compound according to claim 19 for the manufacture of a medical-sanitary product for therapeutic prophylaxis in aging neurology, both against the effects of aging on memory, on other cognitive functions and on modifications of personality in the elderly, and against neurologic diseases correlated to age (cerebral atherosclerosis, adverse reactions to drugs) together with the action against anxiety and depression.

34. Use of a compound according to claim 19 for the manufacture of a medical-sanitary product for therapeutic prophylaxis in the physiopathology of gastroesophageal reflux, interrupting neurologic circuits that can induce depression.

35. Use of a compound according to claim 19 for the manufacture of a medical-sanitary product for therapeutic prophylaxis in the physiopathology of OSAS on depression and anxiety and also by inducing a neuronal functional reset with reduction of central and obstructive apneas.

36. Use of a compound according to claim 19 for the manufacture of a medical-sanitary product for therapeutic prophylaxis in the physiopathology of OSAS on anxiety and depression and to induce an improvement in sexual functions with a functional reset of neuron neurotransmission and hormone release.
